(19) United States
(12) Patent Application Publication
(10) Pub. No.: US 2008/0249395 A1
(43) Pub. Date: Oct. 9, 2008

(54) METHOD AND APPARATUS FOR CONTROLLING CATHETER POSITIONING AND ORIENTATION

(76) Inventors: Yehoshua Shachar, Santa Monica, CA (US); Bruce Marx, Ojai, CA (US); Leslie Farkas, Ojai, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 11/697,690

(22) Filed: Apr. 6, 2007

(51) Int. Cl. A61B 5/05

(52) U.S. Cl. 600/409; 600/424; 600/426

(57) ABSTRACT

A method and apparatus for detecting position and orientation of catheter distal magnetic element end while moving in a patient's heart is described. The apparatus includes magnetic sensors for to detect the magnetic field of a generated by the catheter tip. Each sensor transmits the field magnitude and direction to a detection unit, which filters the signals and removes other field sources, such as generated by CGCI coils and external medical hardware. The method allows the measurements of magnitude corresponding to the catheter tip distance from the sensor and the orientation of the field showing the magnetic tip orientation. Since the tip's magnetic field is not necessarily symmetric, the position and orientation computation technique are not independent of each other. Hence, an iterative calculation is used to converge to a solution. The method of determining tip position is calculated by triangulation from each sensor. In one embodiment, the tip orientation is calculated by an intersecting-planes algorithm. The orientation is used to adjust the distances from each sensor, and the process is repeated until convergence for both position and orientation is achieved. The resultant value provides the actual catheter tip position and orientation (AP). The actual position is further filtered by synchronizing the AP measurements with the QRS signal of the heart, allowing the operator and CGCI controller to view the organ as a static object.

### BACKGROUND

1. Field of the Invention

The invention relates to systems and methods for guiding, steering, and advancing an invasive medical device in a patient while using a sensor and fiducial markers to determine the location and orientation of the catheter.

2. Description of the Prior Art

Catheterization is typically performed by inserting an invasive device into an incision or body orifice. This procedure relies on manually advancing the tip of the invasive device by pushing, rotating, or otherwise manipulating the proximal end which remains outside of the body.

The prior art approach to solving the fiducial relationship between the moving catheter tip and the heart relies on manually advancing the catheter tip by pushing the proximal end of the catheter, while grossly neglecting the respiratory outputs (ribcage displacement), heart contraction, QRS synchronization, patient operating bed location, image capture (x-ray or other imaging modalities) to approximately move the catheter from the current Actual Position (AP) to the Desired Position (DP).

Review of the prior art and its limitations indicate the inability of the prior art solutions to account for the global orientation of the catheter tip relative to frame distortion associated with the operating table, QRS synchronization timing point, or image acquisition, where the data points (e.g., the vector space) are normalized to the patient local coordinate system.

The limitations of the prior art are characterized by the fact that the operator requires the system to define an accurate position for moving the catheter tip from actual position to its desired position in an “autopilot” regiment. The prior art cited can not perform the task because the position and orientation of the catheter tip is influenced by more than the local coordinate system and it is dependent on many variables such as, heart dynamics, fiducial external sensor, patient's body relative to the operating table, etc.

Therefore, there is a substantial and unsatisfied need for an apparatus and method for detecting the position and orientation of a medical tool such as catheter and catheter like devices in order to guide, steer, advance the position of an invasive device and for accurately controlling their position, for providing three dimensional imaging and for minimizing the use of x-ray or other ionizing radiation.

### SUMMARY

The system described herein solves these and other problems by locating the catheter tip in a magnetic chamber and within the patient's body. In one embodiment, the catheter is located in the heart in the presence of dynamic motion under the QRS regiments (e.g., while undergoing the systole/diastole cycle).

The position and orientation of the catheter is tracked in the presence of dynamical variables, such as, for example, movement of the catheter from its actual position (AP) to its desired position (DP), the dynamics of the patient's heart during its mechanical contraction and repolarization of the heart muscle, the location of the catheter tip relative to the organ's specific anatomy, the operating table, and all the above variables relative to the orientation of the imaging modality used in viewing the organ, (e.g., ultrasonics, radar, x-ray, x-ray with different angulation AP caudal 20° etc.).

Many of the variables are relatively independent of each other such that there is no useful functional relationship between each one of the elements which define the position of the catheter so as to predicate the other variables. The operating table position, the respiratory chest positions, the movement of catheter tip from AP to DP, the heart cycle, the QRS signal, and the x-ray image orientation are relatively independent variables. One embodiment provides correlation of these variables.

In a catherization system, the CGCI must be capable of identifying the position and orientation of the catheter tip in order to be able to operate in closed-loop servo mode.

In one embodiment, the magnetic system is further improved by using a shaped magnetic field to reduce the power required to move the catheter tip from the AP to the DP.

In one embodiment, the system is used in connection with a cardiology procedure, such as, for example, an electrophysiological (EP) procedure of mapping and ablation by using the CGCI to control, guide, and image the catheter.

One embodiment includes a servo closed-loop CGCI controller where:
i. The patient's rotation relative to the catheter's tip (the tip) is independent of the transformation for finding the accurate position and orientation of the catheter's tip.
ii. The location and movement of the organ (e.g., heart) relative to the operating table is independent of the transformation for finding the accurate catheter's position and orientation.
iii. The patient's position and heart orientation relative to the operating table as well as the orientation of the auxiliary imaging equipment are independent variables and can be accounted for. Without loss of generality, the system can provide an accurate catheter position and orientation under conditions noted above.
iv. The system is able to provide servo closed loop control while accounting for translation and rotation of the catheter tip relative to a set of independent variables such as the patient's orientation relative to the operating table, image acquisition orientation (e.g., AP, RAO, caudal, etc), respiration mode, and including the QRS heart dynamic (muscle contraction and repolarization).
v. The system, including the catheter position detection unit and its three dimensional vectors, the QRS synchronization unit, the fiducial alignment system, the operation console (includes the display, haptic controller, and mouse) the mapping unit, the position recording unit, the fiducial sensor, the operating table, configuration file, holding the three dimensional models, three dimensional heart model, three dimensional torso model, three dimensional atrial parts model) determines the position and orientation of the catheter tip under dynamic conditions as indicated in i thru iv above. The patient's organ and its specific anatomical site are synchronized to form a normalized vector field (orthogonal to the global coordinate system) further facilitating the operation of the servo closed loop

One embodiment includes a CGCI apparatus for determining the position and orientation of the catheter's tip under an translation or rotation of variables such as: patient specific anatomical features such as the heart, right atrium, inferior vena cava, superior vena cava, right atrium lateral wall, HIS bundle, interatrial transseptum, heart left atrium, heart right ventricle, heart left ventricle, heart tricuspid valve, heart mitral valve, electrocardiac signal, QRS synchronization timing point, and respiration signal, etc.

The catheter tip is detected and displayed relative to the fiducial sensor position and orientation with its orthogonal vector set: the fiducial x-axis, FX; fiducial y-axis, FY; fiducial z-axis, FZ; and fiducial position, FP.

In one embodiment, the actual catheter tip and virtual catheter tip are normalized under the global orientation transformation matrix, GO, and global position transform matrix, GP.

In one embodiment, the detection unit provides data for defining the location of the catheter tip, forming a map and synchronizing the patient, his or her heart and specific anatomical features e.g. HIS bundle, its electrocardiac signal, and/or its QRS synchronization timing point. The catheter position and orientation is further corrected relative to respiration signal (pulmonary chest displacement).

The closed loop control system uses the ability of the imaging and synchronization module to locate, identify, and report the position and orientation of the catheter tip in three dimensional space under dynamic conditions (e.g., heart muscle contraction and repolarization) while considering respiration distortion of the ribcage, the patient's position relative to the operating table and the specific image capture relative to the patient's organs.

The system provides an accurate position and orientation of the catheter's tip the presence of relatively independent dynamic variables. The movement of the catheter tip from its actual position AP (catheter axis, global coordinates, and catheter position, global coordinate) to its desired position DP, the movement of the catheter tip, the patient, heart and its specific anatomical features (e.g. heart left atrium) are relatively independent variables.

In one embodiment, the system is used in connection with a procedure for finding the tricuspid valve in a rotated patient. In one embodiment, the system is used in connection with a procedure for finding the pulmonary vein in a rotated patient.

In one embodiment, the system is configured to determine the position and orientation of the catheter tip and or surgical tools while accounting for mechanical contraction of the heart muscle, its electrical excitation propagation in three dimensional space (one embodiment of a technique for computing the Laplacian cardiac electrogram and the wave equation characteristics used by the CGCI apparatus is further described by U.S. patent application Ser. No. 11/362,542, hereby incorporated by reference).

In one embodiment, the multiple dynamic and independent variables such as QRS complex, catheter position and orientation, and the outside fiducial markers are normalized in real time to facilitate the servo closed loop modality for controlling the movement of the catheter from AP to a DP. The actual position (AP) is mapped onto the virtual models and patient anatomy by using the global position and orientation matrices generated so as to account for the shifts of position and orientation of the Fiducial alignment sensor caused by patient motion and ribcage displacement due to respiration. Using these matrices, the operator commands, the patient and the patient data are synchronized, and the desired position and orientation (DP), is generated as a conformal map relative to the actual catheter tip position (AP) and patient anatomy, further forming a servo close loop modality for Control, Guidance and Imaging of catheter tip in a magnetic chamber.

In one embodiment, a fiducial map is used to relate dimensional, anatomical and electrical elements in real time.

One embodiment includes a magnetic catheter guidance and control apparatus that requires less training and less skill than prior art systems. In one embodiment, a radar system is used to determine the location of the tip of the catheter inside the body, thus minimizing or eliminating the use of ionizing radiation such as X-rays. Alternatively, the catheter guidance system can be used in combination with an X-ray system (or other imaging system) to provide additional imagery to the operator. Moreover, the magnetic system used in the magnetic catheter guidance system can also be used to locate the catheter tip to provide location feedback to the operator and the control system. In one embodiment, a magnetic field source is used to create a magnetic field of sufficient strength and orientation to move a magnetically-responsive catheter tip in a desired direction by a desired amount.

One embodiment includes a catheter and a guidance and control apparatus that can accurately, and with relative ease, allow the surgeon/operator to position the catheter tip inside a patient's body. The catheter guidance and control apparatus can maintain the catheter tip in the correct position. One embodiment includes a catheter and a guidance and control apparatus that can steer the tip of the catheter through arteries and forcefully advance it through plaque or other obstructions. One embodiment includes a catheter guidance and control apparatus that displays the catheter tip location with significantly reduced X-ray exposure to the patient and staff One embodiment includes a catheter guidance and control apparatus that is more intuitive and simpler to use, that displays the catheter tip location in three dimensions, that applies force at the catheter tip to pull, push, turn, or hold the tip as desired, and that is capable of producing a vibratory or pulsating motion of the tip with adjustable frequency and amplitude to aid in advancing the tip through plaque or other obstructions. One embodiment provides tactile feedback at the operator control to indicate an obstruction encountered by the tip.

In one embodiment, the catheter guidance control and imaging (CGCI) system allows a surgeon to advance, accurately position a catheter, and to view the catheter's position in three dimensions by using a detection system to locate the tip of the catheter. In one embodiment, the detector data can be combined with X-ray imagery to produce a composite display. In one embodiment, the detector is a system which includes a synthetic aperture radar (SAR). In one embodiment, the radar system includes an ultra-wideband radar. In one embodiment, the radar system includes an impulse radar.

In one embodiment, the apparatus includes a user input device called a “virtual tip” that, in addition to being a representation of the actual or physical catheter tip advancing within the patient's body, possesses a positional relationship to the catheter tip. The virtual tip includes a haptic joystick that can be manipulated by the surgeon/operator and is also designed to deliver tactile feedback to the surgeon in the appropriate axis or axes if the actual tip encounters an obstacle. In other words, the virtual tip includes a joystick-type device that allows the surgeon to guide the actual catheter tip though the patient's body. When the actual catheter tip encounters an obstacle, the virtual tip provides tactile force feedback to the surgeon to indicate the presence of the obstacle.

In one embodiment, the physical catheter tip (the distal end of the catheter) includes a permanent magnet that responds to a magnetic field generated externally to the patient's body. The external magnetic field pulls, pushes, turns, and holds the tip in the desired position. One of ordinary skill in the art will recognize that the permanent magnet can be replaced or augmented by an electromagnet.

In one embodiment, the physical catheter tip (the tip of the catheter) includes a permanent magnet and two piezoelectric rings, or semiconductor polymer rings to allow the detector to sense the second harmonics of the resonating signal emanating from the rings.

In one embodiment, the CGCI apparatus uses a technique of image synchronization by using a sensor having six degrees of freedom (6-DOF), thereby allowing the formation of a stereotaxic frame of reference.

In one embodiment, the electromagnetic circuit of the CGCI apparatus includes a C-arm geometry using a ferromagnetic substance (e.g., a ferrite substance) so as to increase the efficiency of the magnetic circuit.

In one embodiment, the CGCI apparatus uses numerical transformations to compute currents to be provided to various electromagnets to control the magnetic field used to push, pull and rotate the catheter tip in an efficient manner.

In one embodiment, the CGCI apparatus includes an ultrawideband (UWB) impulse radar and a 6-DOF sensor configured to detecting the catheter tip and moving body organs, and synchronize their motions.

In one embodiment, the CGCI apparatus is used to perform an implantation of a pace-maker leads during an electrophysiological (EP) procedure.

In one embodiment, the CGCI apparatus uses a detector or other sensors to measure, report and identify the location of a moving organ within the body (e.g., the heart, lungs, etc), with respect to the catheter tip and one or more fiducial markers, so as to provide guidance control and imaging to compensate for movement of the organ, thereby simplifying the surgeon's task of manipulating the catheter through the body.

In one embodiment, the operator control provides the position and orientation command inputs to a servo system that controls the catheter tip position by regulating the magnetic force applied outside the patient's body. A measurement of the actual tip position and orientation is made via sensory apparatus that includes a radar system, and the 6-DOF sensor. This measurement is used to provide feedback to the servo system and the operator interface. In one embodiment, the servo system has a correction input that compensates for the dynamic position of a body part, or organ, such as the heart, thereby offsetting the response such that the actual tip moves substantially in unison with the beating heart.

In one embodiment, operation of the catheter guidance system is as follows: i) the operator adjusts the physical position of the virtual tip, ii) a change in the virtual tip position is encoded and provided along with data from the detector system and a 6-DOF sensor to a control system, iii) the control system generates servo-system commands that are sent to a servo system control apparatus, iv) the servo system control apparatus operates the servo mechanisms to adjust the force (B) of one or more electromagnet clusters. The position of the actual magnetic catheter tip within the patient's body to change, v) the new position of the actual catheter tip is then sensed by the detector and the position of a plurality of fiducial markers are sensed by the 6-DOF sensor, thereby allowing synchronization and superimposing of the catheter position on an image produced by fluoroscopy and/or other imaging modality, and vi) providing feedback to the servo system control apparatus and to operator interface and updating the displayed image of the actual catheter tip position in relation to the patient's internal body structures.

The operator can make further adjustments to the virtual catheter tip position and the sequence of steps ii through vi are repeated. In one embodiment, feedback from the servo system control apparatus creates command logic when the actual catheter tip encounters an obstacle or resistance in its path. The command logic is used to control stepper motors which are physically coupled to the virtual catheter tip. The stepper motors are engaged as to create resistance in the appropriate directions that can be felt by the operator, and tactile feedback is thus provided to the user.

### BRIEF DESCRIPTION OF THE DRAWINGS

FIG. 1 is a block diagram of the CGCI unit showing the imaging and synchronization subsystem and its function in obtaining AP and specifying DP.

FIG. 2 is a first perspective view of the catheter tools.

FIG. 3 is a second perspective view of the catheter tools.

FIG. 4 is a third perspective view of the catheter tools.

FIG. 5 is a fourth perspective view of the catheter tools.

FIG. 5 is an illustration of the magnetic chamber used in the CGCI Controller.

FIG. 6 shows the operator console screen showing the command function and data blocks.

FIG. 7 shows the operator console screen showing the position reference data and position reference markers.

FIG. 8 shows the operator console screen showing the command functions and visual references objects.

FIG. 9 shows a haptic controller and its coordinate system.

FIGS. 10 and 10A are block diagrams of the Catheter Detection Unit, its basic internal routines and its function in providing catheter global coordinates, AP, to the fiducial system.

FIG. 11 is a schematic diagram of a Hall-Effect sensor in relationship to the catheter tip, and its generated measurement data.

FIG. 12 is a schematic diagram of a catheter position triangulation using four Hall-Effect sensors and their respective range values.

FIG. 13 is a detailed schematic of the triangulation of the catheter position's x-coordinate using two sensors.

FIG. 14 is a diagram of the Intersection Planes Method for determining the orientation of a catheter tip from its position and two sensor's magnetic field values.

FIGS. 15 and 15A are illustrations of the fiducial system's function in relation to breathing movements.

FIG. 16 is a schematic of the Fiducial Alignment System's circuitry.

FIG. 17 is a schematic diagram of the relationship between local, global, and fiducial coordinate systems and their associated reference vectors.

FIG. 18 is the general form of the matrix used to rotate one vector about another by a given angle in a left-handed coordinate system.

FIG. 19 is a reproduction of the operator console screen for Fiducial Example 1, illustrating the catheter tip's placement in the Tricuspid Valve of an unrotated patient, and highlighting the corresponding data.

FIG. 20 is a reproduction of the operator console screen for Fiducial Example 1, illustrating the catheter tip's placement in the Tricuspid Valve of a rotated patient, and highlighting the corresponding data.

FIG. 21 is a reproduction of the operator console screen for Fiducial Example 2, illustrating the catheter tip's placement in the pulmonary vein of an unrotated patient, and highlighting the corresponding data.

FIG. 22 is a reproduction of the operator console screen for Fiducial Example 2, illustrating the catheter tip's placement in the pulmonary vein of a rotated patient, and highlighting the corresponding data.

### DETAILED DESCRIPTION

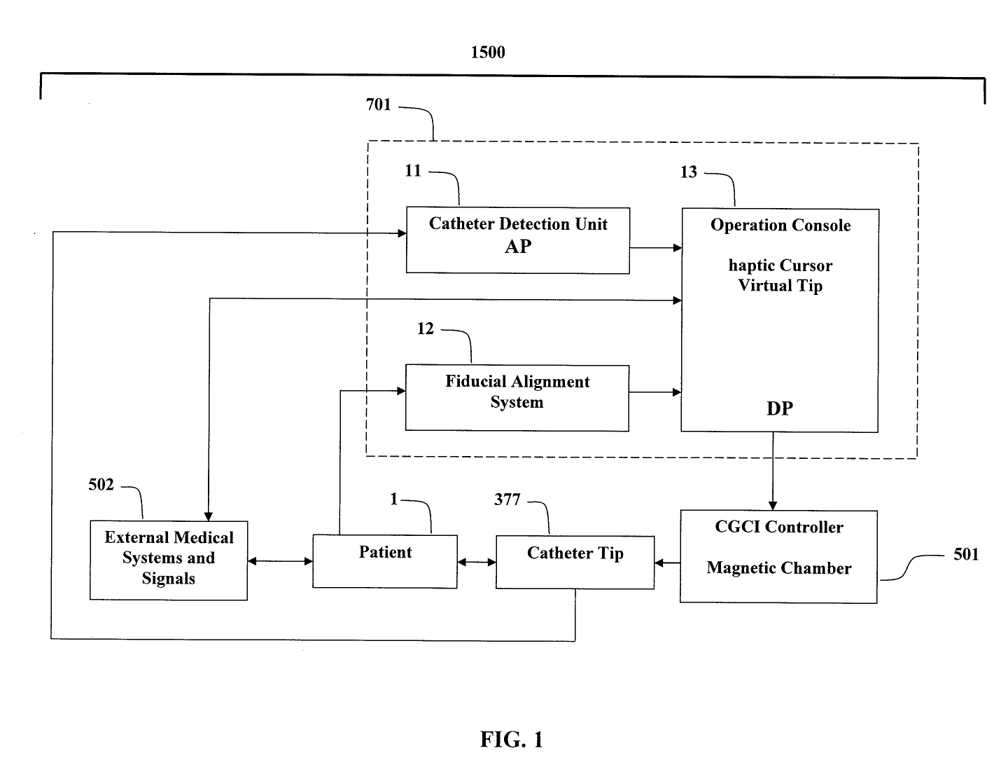

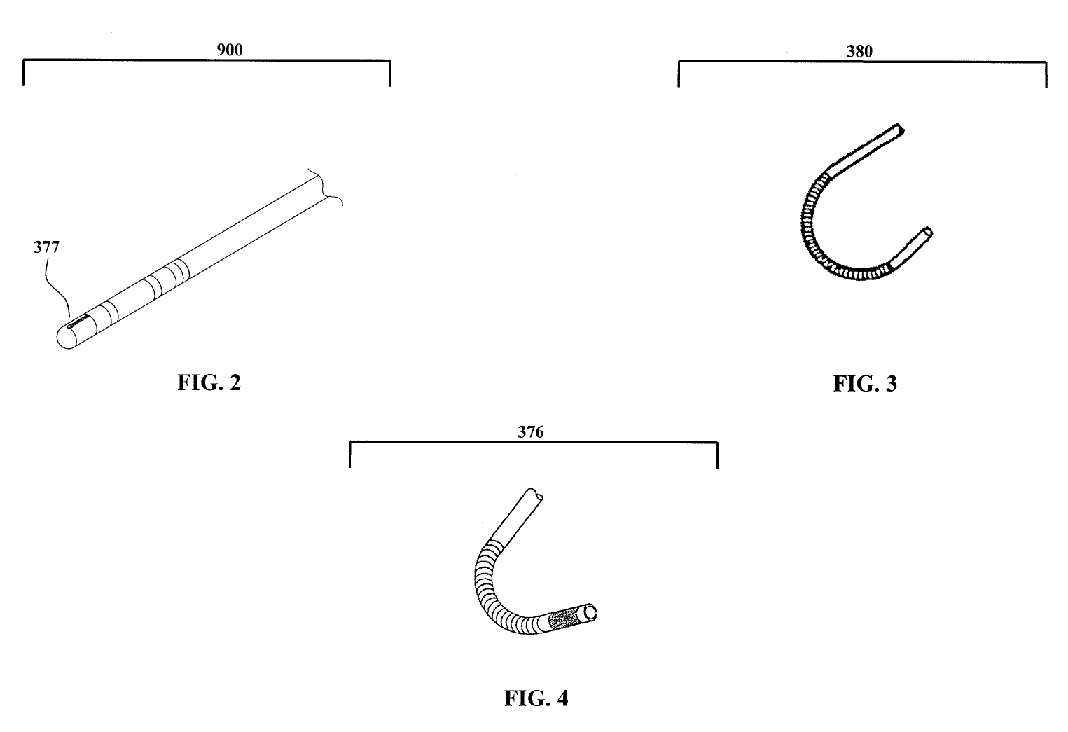

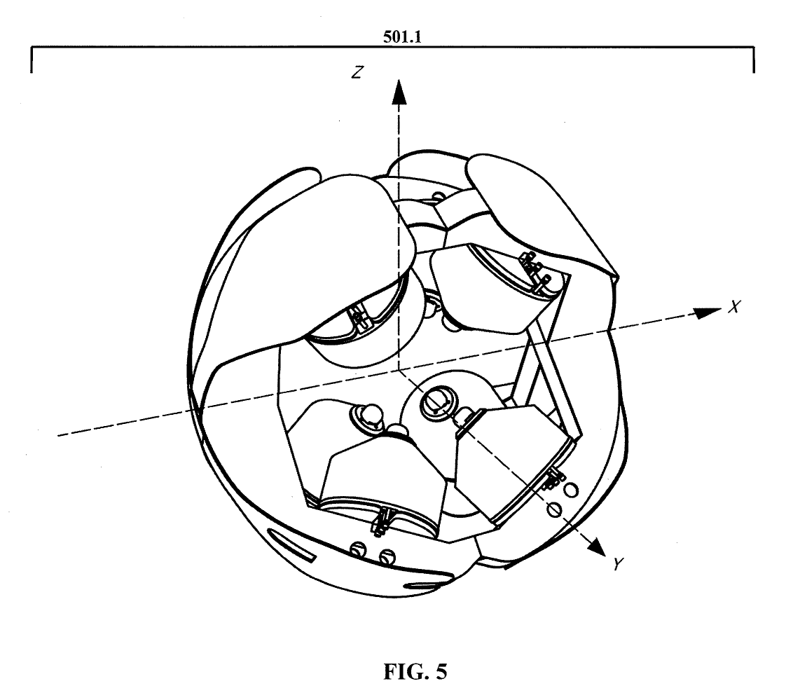

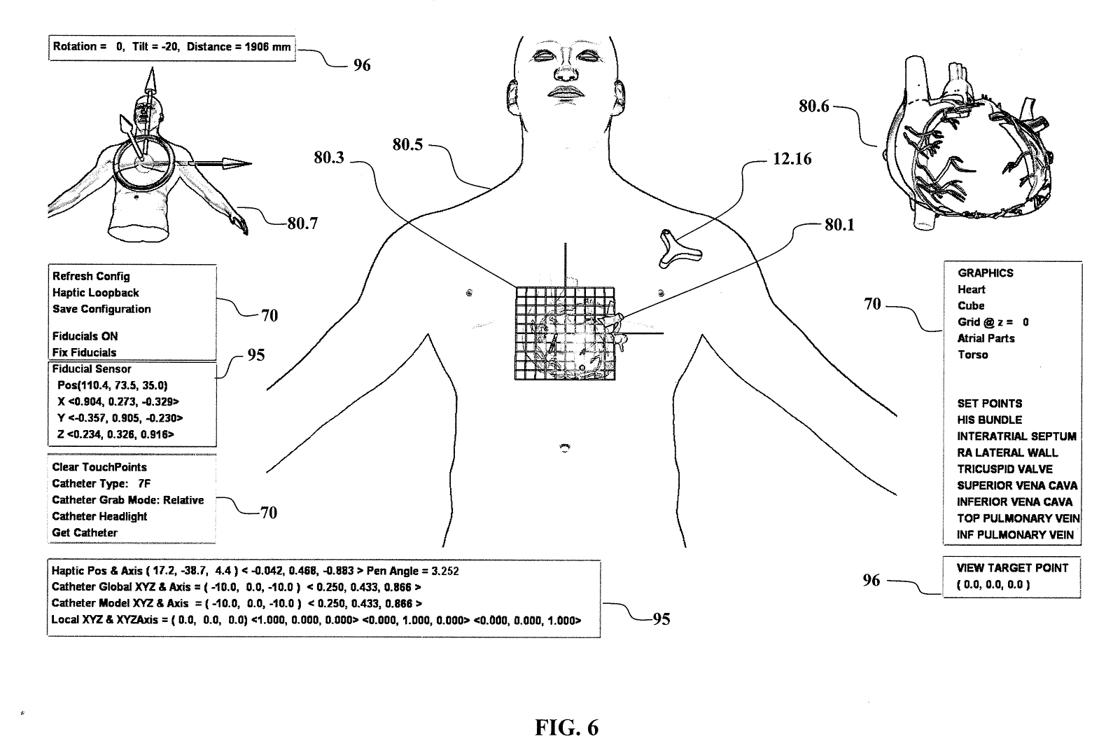

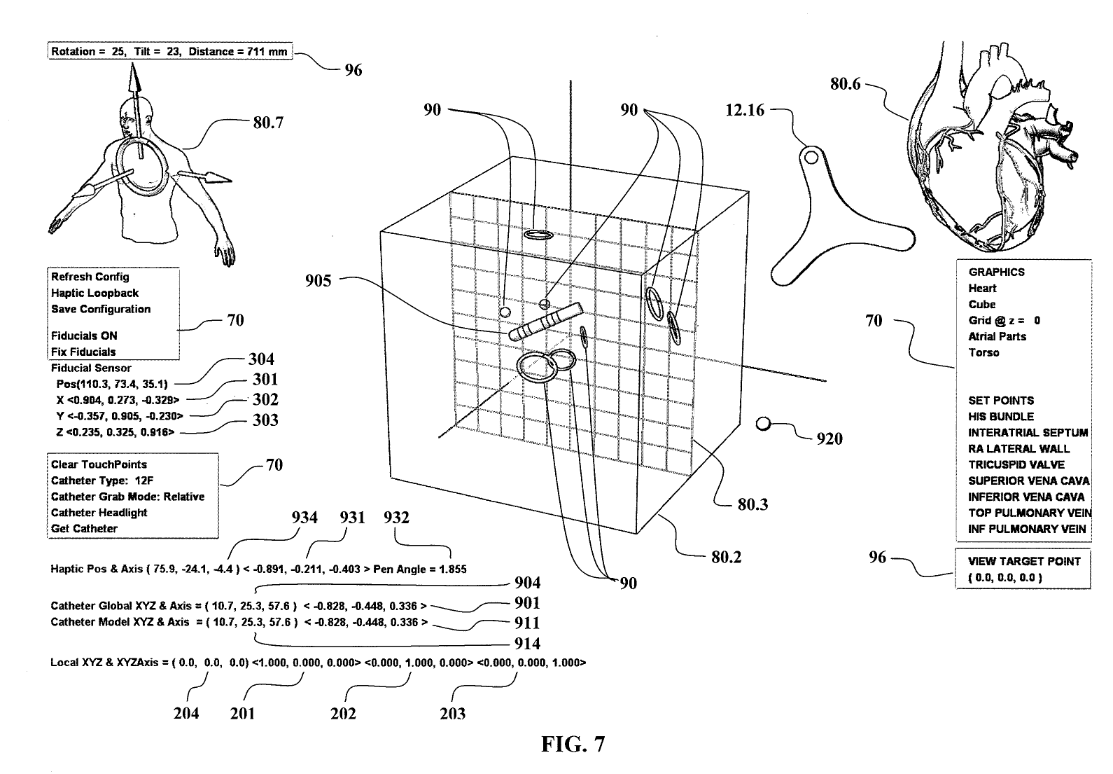

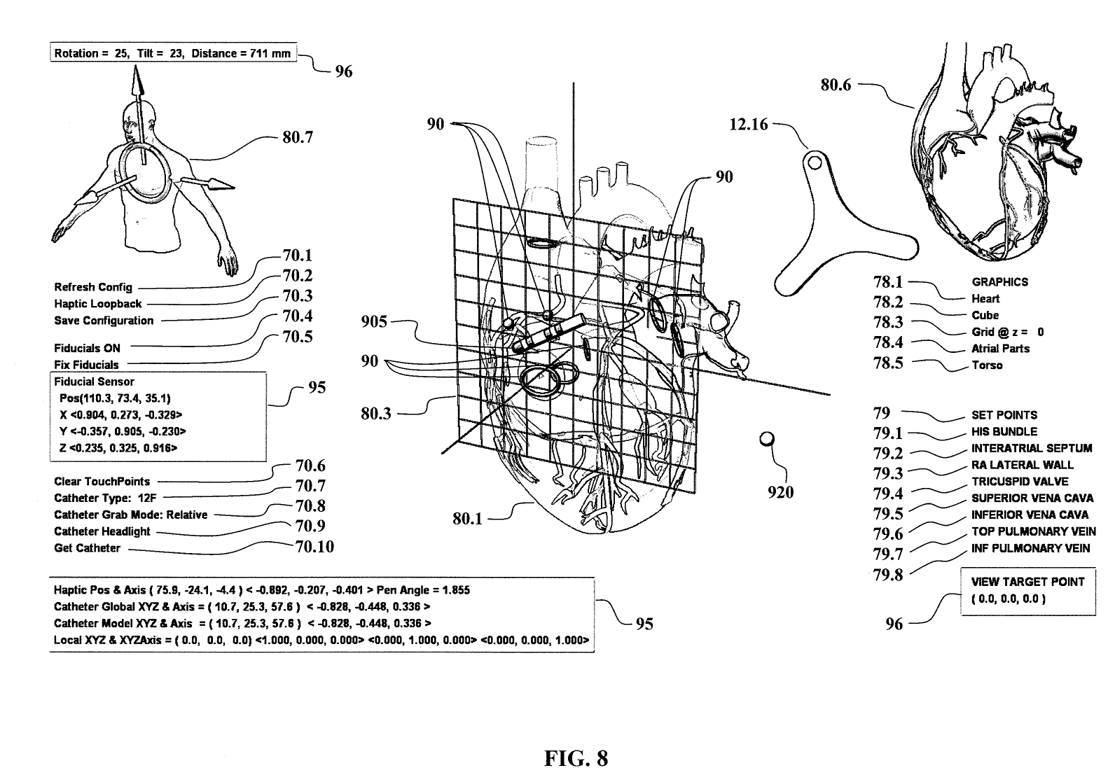

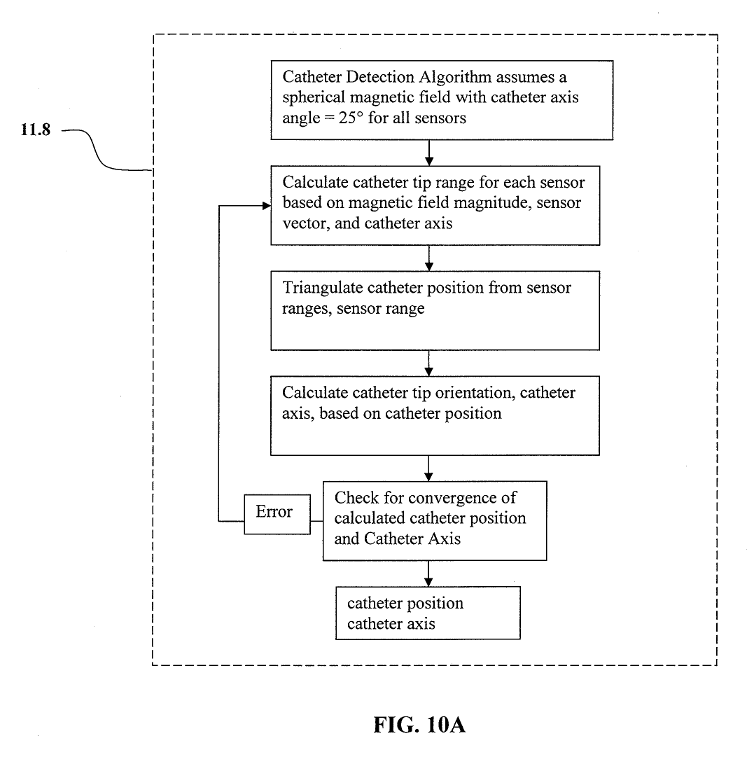

FIG. 1 is a block diagram of a CGCI unit 1500 what includes an imaging and synchronization unit 701, a catheter detection unit 11, a fiducial alignment system 12, and an operation console 13. In the CGCI Unit 1500, the operator navigates a magnetically tipped catheter 377 within a patient 1 using a six-degree of freedom haptic joystick 18 (shown in FIG. 9) while visualization of the progress of a virtual catheter tip 905 within the operation console's three dimensional virtual environment 13 as shown in FIG. 6. The catheter tip position detection system 11 provides the current position of the catheter tip or actual position (AP) 902, and the operator's position movement commands to move the catheter to a desired position (DP) 903 are sent to the catheter tip position control system or the CGCI controller 501 in FIG. 1.

Use of a magnetic chamber with an adaptive regulator, while using a joystick haptic device for operator control and method for detecting a magnetically tipped catheter is described in U.S. patent application Ser. No. 10/621,196 “Apparatus for Catheter, Guidance, Control, and Imaging38 hereby incorporated by reference. The technique and apparatus is further explained by U.S. patent application Ser. No. 11/33 1,78 1, “System and Method for Controlling Movement of a Surgical Too.” hereby incorporated by reference. The magnetic apparatus for generating controlled movement in the patient's body is detailed by U.S. application Ser. No. 11/331,994, “Apparatus and Method for Generating a Magnetic Field” hereby incorporated by reference. The method for controlling a surgical tool within the patient's body and the technique for such use is described by U.S. application Ser. No. 11/331,485, “System and Method for Magnetic Catheter tip,” “System and Method for Radar Assisted Catheter Guidance and Control” U.S. application Ser. No. 10/690,472, titled, “System and Method for Radar Assisted Catheter Guidance and Control,” hereby incorporated by reference, describes a method for detecting the catheter in a patient's body by using a sensor array such as radar and Hall Effect sensor combined with a set of fiducial markers,. The magnetic chamber, its geometry and the formation of servo closed loop is detailed by U.S. application Ser. No. 11/140,475, “Apparatus and Method for Shaped Magnetic Field Control for Catheter, Guidance, Control and Imaging.” The use of a magnetically tipped catheter while guided, controlled, and imaged by the apparatus noted above, is used in cardiology and specifically while mapping the electrical characteristics of the human heart to allow a controlled, accurate and efficient delivery of ablating RF energy. The use of the CGCI method and apparatus detailed above for use in electrophysiological mapping and ablation is noted by U.S. application Ser. No. 11/362,542, “Apparatus for Magnetically Deployable Catheter with Mosfet Sensors and Method for Mapping and Ablation.”

In one embodiment, the CGCI (Catheter, Guidance, Control, and Imaging) system advances and controls the catheter tip using a servo closed loop where the magnetic field in the chamber is controlled produce translation and rotation of the catheter tip within the magnetic chamber. This process of controlling the catheter-tip in magnetic chamber is dependent on the ability of the CGCI apparatus to detect, calculate, and define accurately and in real-time the position and orientation of the catheter tip.

The system 1500 allows a surgeon to move a catheter and other invasive tools within the patient's body while accounting for catheter position and orientation coordinates:
i. while moving from AP to DP,
ii. where the heart dynamics (systole, and diastole) are gated during movement of the catheter tip from AP to DP,
iii. is defined while the rib cage displacement during respiratory cycle is accounted for during the process of establishing the tip of the catheter tip,
iv. is determined relative to the auxiliary imaging apparatus orientation to allow the orthogonal representation of the image capture with the specific anatomical feature of the patient's heart relative to the position and orientation of the catheter tip,
v. Is coupled with the image (from x-ray, or other imaging modalities) to gate such coordinates with the patient's heart electrical activity, QRS,
vi. to account for global transformation relative to the local transformation of any of the above mentioned variables while the catheter tip translation and/or rotation are fixed relative to each other, and
vii. to inform a controller to form a servo closed loop modality for manipulating the catheter tip from AP to DP while accounting for the dynamic state of the independent variables noted above.

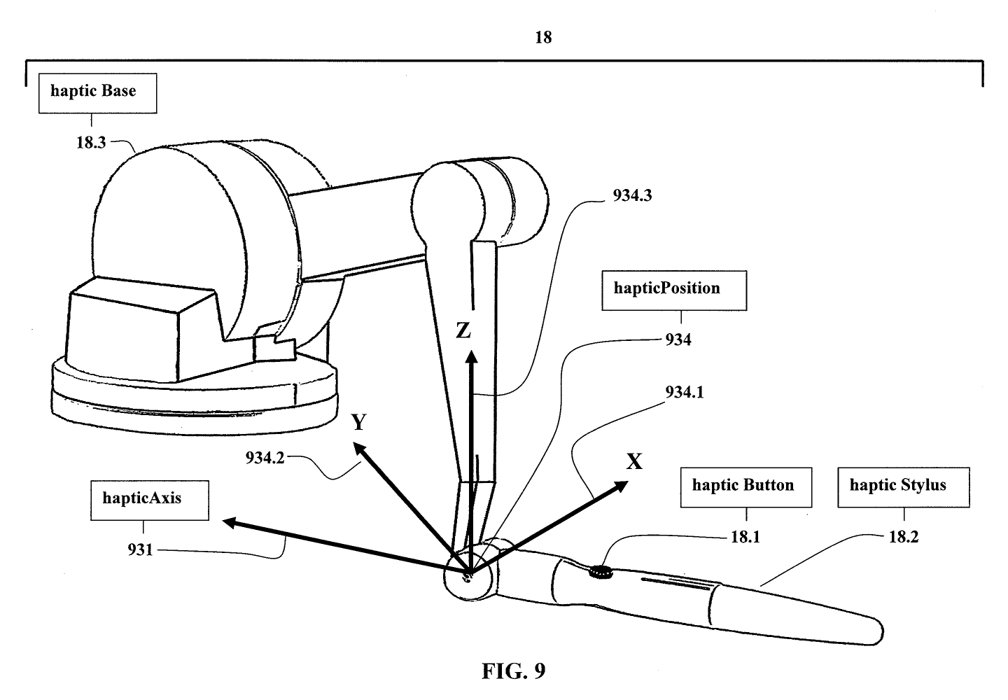

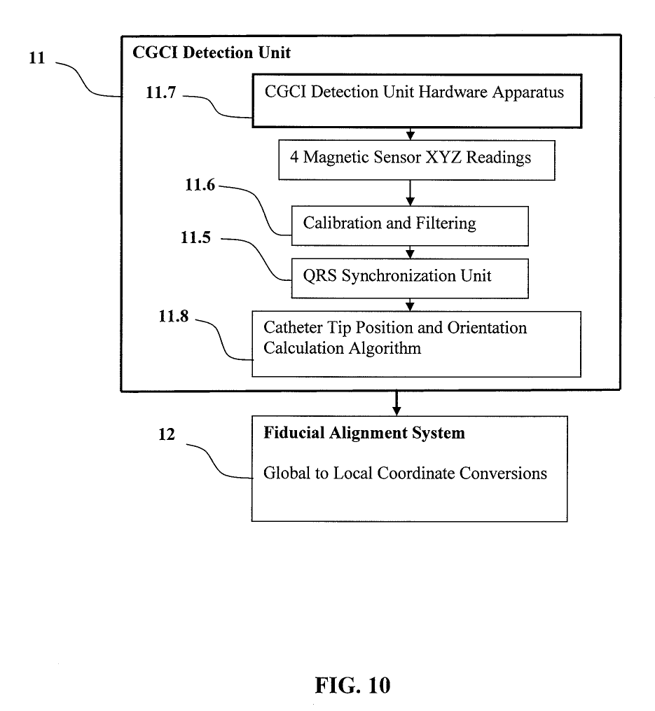

The operation console 13 in FIG. 1 includes the display screens (which produce displays as shown, for example in FIGS. 6-8 and 19-22), the haptic joystick 18 shown in FIG. 9, and a mouse (not shown). In the displays, the virtual catheter tip 905 in FIG. 7 is shown in relation to the user-selected three dimensional anatomical models 80. The view can be rotated and zoomed in and out for the proper perspective. Additional navigational reference icons 80.7 and 80.6 show the global view of the patient 1 and the local view of the heart respectively. The mouse-selectable command buttons 70 allow the operator to turn on or off graphical objects 80, customize the haptic cursor 920 in FIG. 7 and lay down position reference markers 90 within the virtual models 80. Position reference data 95 in FIG. 8 is also displayed as needed by the operator.

The haptic joystick controller 18 in FIG. 9 is used to command the catheter tip 377. The system causes the catheter tip to follow movements of the virtual catheter tip 905 in FIG. 7. When the virtual tip is moved, a new desired position (and orientation), DP, is sent to the CGCI controller 501 in FIG. 1. The controller 501 controls the electromagnets of the system 1500 to move the catheter tip 377 to the new position and orientation, DP. If the position and orientation, DP, cannot be obtained by the CGCI controller 501, the haptic joystick 18 forces the haptic stylus 18.2 to provide tactile feedback to the operator using internal motors, informing the operator that the location is blocked (an obstacle is encountered). This allows the operator to sense the contours and blockages of the patient's anatomy 1 and prevent to the patient. In one embodiment, the amount of force provided as tactile feedback is computed as a function of the error between the desired position and the actual position. In one embodiment, the amount of force provided as tactile feedback is computed as a function of the error between the desired orientation and the actual orientation of the tip. The imaging and synchronization unit 701 further allows the operator to view the beating heart by using a synchronizing technique, gating the QRS heartbeat signal, and allowing the operator to sense a moving heart as a static object.

A fiducial sensor 12.16 (shown in FIG. 7) is located on the patient's chest, and keeps the virtual models aligned with the patient's position and orientation relative to such objects as the table, auxiliary imaging equipment, etc. The fiducial alignment system 12 in FIG. 1 is fixed in relationship to virtual models 80, providing the imaging and synchronization unit 701 with a fixed frame of reference relative to the catheter tip's current position and orientation, AP 902. The fiducial alignment system 12 tracks the patient's motion (rotation and translation) synchronously with the virtual models 80. The fiducial alignment system 12 further allows the virtual catheter tip 905 to refer back to the unrotated patient and virtual models 80 to allow the operator/surgeon to accurately relate to the patient's anatomy and add new data to the original imagery.

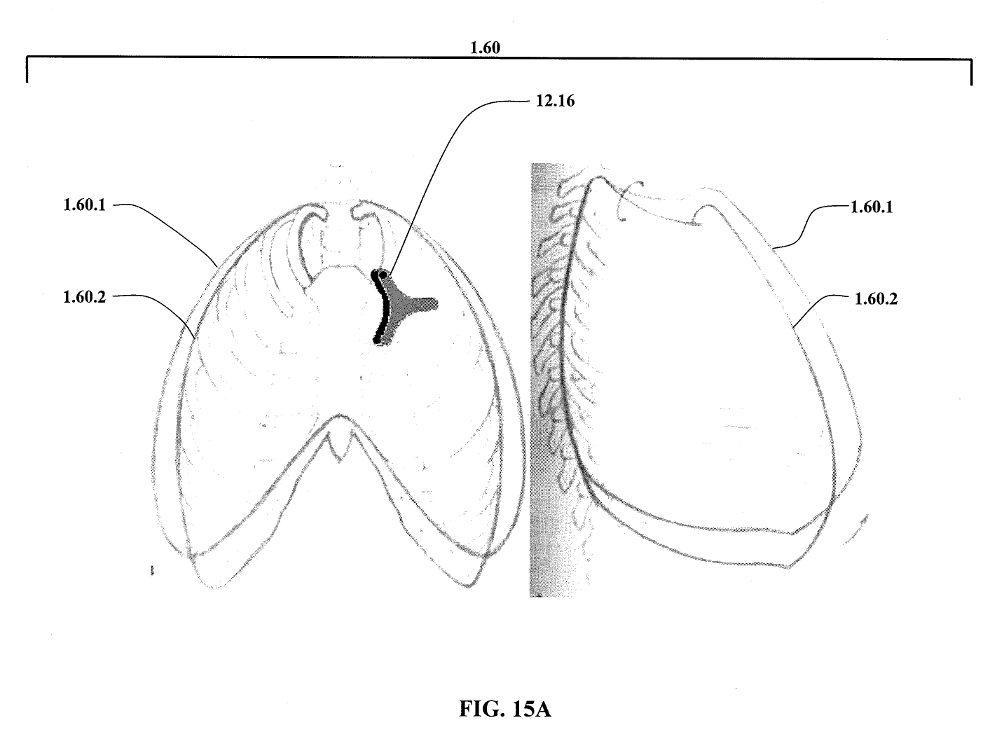

A procedure using the CGCI 1500, such as ablation in the left pulmonary vein, includes aligning the patient 1 on the operating table and attaching the fiducial sensor 12.16 to the patient's chest. A guide wire is inserted in the right femoral vein and guided into the right atrium via the inferior vena cava. A perforation is created in the wall between the right atrium and left atrium forming the interatrial transseptum. The guide wire is inserted into the left atrium via the interatrial transseptum and a sheath is placed over the guide wire's proximal end and guided into the left atrium. The guide wire is removed from the sheath, and a magnetically tipped catheter is inserted into the sheath and guided into the left atrium. The actual position of the catheter tip, AP 902, is now constantly monitored by the catheter detection unit 11 and displayed in the operator console screen as the virtual catheter tip 905. The operator uses the haptic controller 18 to move the virtual tip 905 to command the CGCI controller 501 in FIG. 1 to move the magnetic tip 377 to a desired position and orientation, DP 903. Using the mapping unit 14 in FIG. 15, the operator builds a virtual model of the left atrium, and places position reference icons 90 at key locations. The operator returns to the right pulmonary veins, positions the catheter tip 377, and begins a standard ablation procedure about the outside edges of the veins.

FIG. 6 is a representation of the operator console screen. The operation console 13 includes the screen, the haptic joystick 18 and the mouse. The central three-dimensional models, including the torso 80.5, the grid 80.3 and the three dimensional heart model 80.1, can be selected using the command buttons 70. The user's viewpoint is designated by the view data 96 and changes with rotation and zoom using the mouse. Additional model icons are provided as a position and orientation reference. The heart local reference icon 80.6 gives the current viewpoint of the complete organ. The global axis and patient reference icon 80.7 displays the orientation of the global coordinates and the patient's relationship to the global coordinates as depicted in FIG. 6. The fiducial sensor 12.16 is shown on the patient and its current position and orientation are displayed in the console data 95.

FIG. 7 further depicts the 100 mm cube representing the central workspace 80.2, and the position reference set points 90 representing key anatomical locations. The virtual catheter tip 905 is shown in relationship to the rest of the geometry, and its global axis 901, global position 904, model local axis 911, and local position 914 in FIG. 7 are presented in the console data display 95 in FIG. 6. The global position 904 described in terms of x,y,z coordinates in the global coordinate space, and the global axis is described in terms of a vector direction. The local position 904 described in terms of x,y,z coordinates in a local coordinate space, and the global axis is described in terms of a vector direction.

The six-degree of freedom haptic controller's cursor 920 is used to grab the virtual catheter tip 905. The haptic cursor's global position 934, global axis 931, and the global angle of rotation of the haptic pen 932 in FIG. 7 are displayed in the console data display 95 in FIG. 6.

The patient and three dimensional model's local coordinate system is a Cartesian coordinate system and has its local position 204, local X-axis 201, local Y-axis 202, and local Z-axis 203 in FIG. 7 displayed in the console data 95 in FIG. 6.

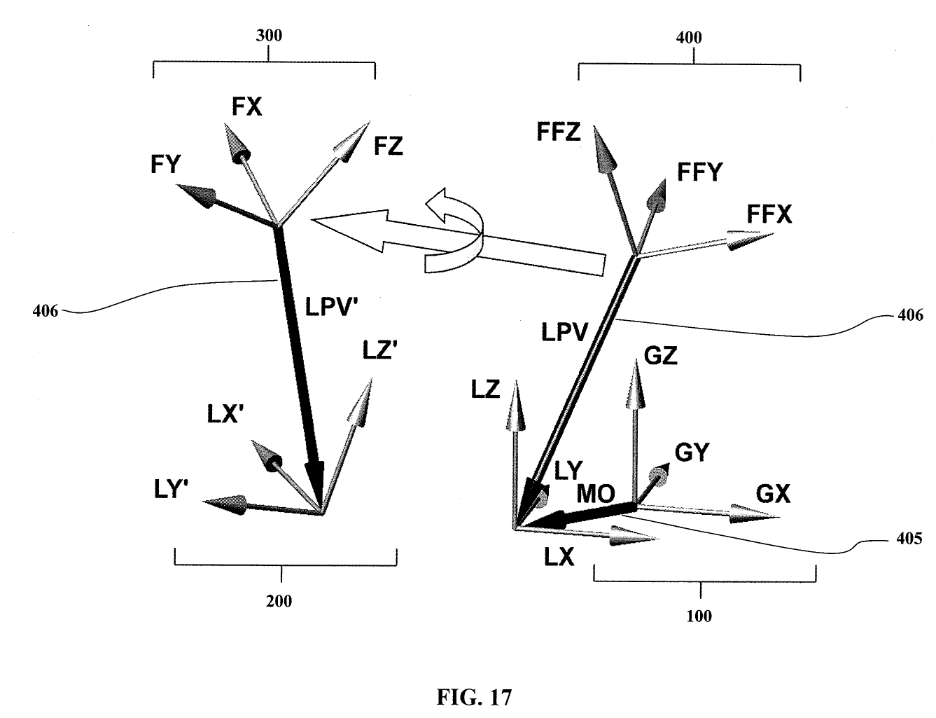

The global coordinate system 100 is also a standard Cartesian coordinate system as shown in FIG. 17, and has the global position origin at (0, 0, 0), the global X-axis set to (1, 0, 0), global Y-axis, in the direction of the patient's head, set to (0, 1, 0), global Z-axis, in the vertical direction (0, 0, 1). This is only displayed in the form of the global axis and patient reference icon 80.7.

The fiducial sensor's 12.16 globally referenced fiducial position 304, fiducial axis X 301, fiducial axis Y 302, and fiducial axis Z 303 in FIG. 7 are also displayed in the console data 95 in FIG. 6.

FIG. 8 further expands on FIG. 7 by providing details of the command buttons 70. The command buttons that control the display of graphical objects can toggle on and off the display of the heart 78.1, cube 78.2, atrial parts model 78.4, and the patient's torso 78.5. The grid button 78.3 changes the z-coordinate elevation of the grid graphic 80.3 providing the operator/surgeon with the ability to further discern the location of the catheter tip 377 relative to the anatomical models 80 or turns the grid off

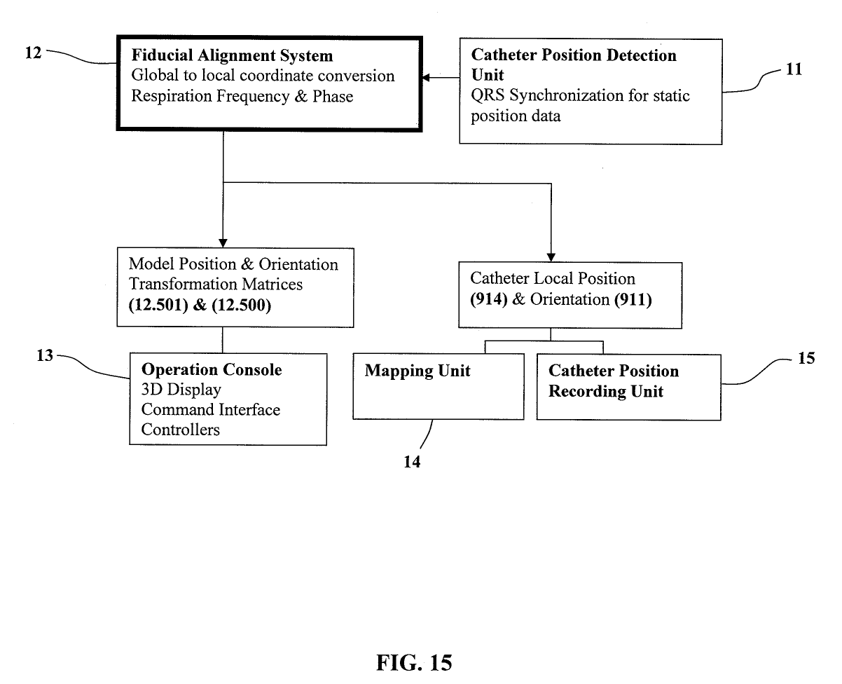

The set points button 79 toggles the display of the position reference set points 90. The set point type and label for each of the set point buttons 79.1-79.8 and position reference set points is set in the configuration file. A mouse right-click on a set point button gives that set point the current catheter tip position 904 and orientation 901 in FIG. 14. A mouse left-click on one of the set point buttons issues a command to guide the catheter tip 905 to that set point's position and orientation. The features noted above allow the operator/surgeon to enhance his visual view of the specific anatomical detail so as to provide a realistic map for performing its intended task.

The system settings are loaded at startup from the configuration file (not shown). The current configuration, including the graphics options 78.1-78.5, the set points 79-79.8, and catheter type 70.7, can be saved to the configuration file with the save configuration button 70.3. The system can be set back to the last saved configuration with the refresh configuration button 70.1.

The haptic loopback button 70.2 sets the system into an internal test mode where the haptic cursor 920 directly controls the catheter position 904 and catheter axis 901. In normal mode, with the haptic loopback button 70.2 off, the haptic controller 18 requests that the CGCI controller 501 move the catheter tip 905 to the haptic global position 934 and haptic global axis 931 in FIG. 9. If the catheter tip 905 is out of grabbing range of the haptic cursor 920, the “get catheter” button 70.10 commands the catheter tip 905 to the current haptic cursor position. The catheter grab mode button 70.8 toggles between a relative grab mode and an axial grab mode. Both grab modes directly command the catheter tip position 904, but command the catheter tip orientation 901 differently. The axial grab mode commands the catheter tip 905 to rotate to the haptic axis 931. The relative grab mode commands the rotation of the catheter tip 905 relative to the rotation of the haptic cursor 920 after the haptic button 18.1 in FIG. 9 is pressed. The catheter headlight button 70.9 turns on or off a virtual guiding catheter headlight displayed on the console screen as a light from the catheter tip 905 to more easily navigate within the virtual models. The catheter type button 70.7 toggles between the preset tool types.

The clear touch points button 70.6 reverts all set points 90 to a neutral position and eliminates all modifications to morphable models, initializing the system to begin the mapping unit 14.

The fix fiducials button 70.5 toggles the fiducial system 12 “on” and “off”. When the fiducial system 12 is turned on, it is initialized and begins to operate. When the fiducial system is turned off, the local coordinate system 200 is given the values of the global coordinate system 100 and the fiducial sensor no longer controls the local coordinate system 200. The fiducial's on/off button 70.4 toggles the display of the fiducial sensor 12.16.

FIG. 9 is an isometric representation of the haptic controller 18 showing its ability to define position and orientation within a 6 degree of freedom environment. The haptic controller includes the haptic stylus 18.2, the haptic button 18.1, and the haptic base 18.3. The haptic stylus is used to specify the haptic position 934 and haptic axis 931 of the haptic cursor 920 with six degrees of freedom. The haptic button 18.1 is used to issue commands via the haptic cursor 920. The individual components of the haptic position, haptic position.x, haptic position.y, and haptic position.z are defined with respect to the haptic controller as shown in FIG. 9.

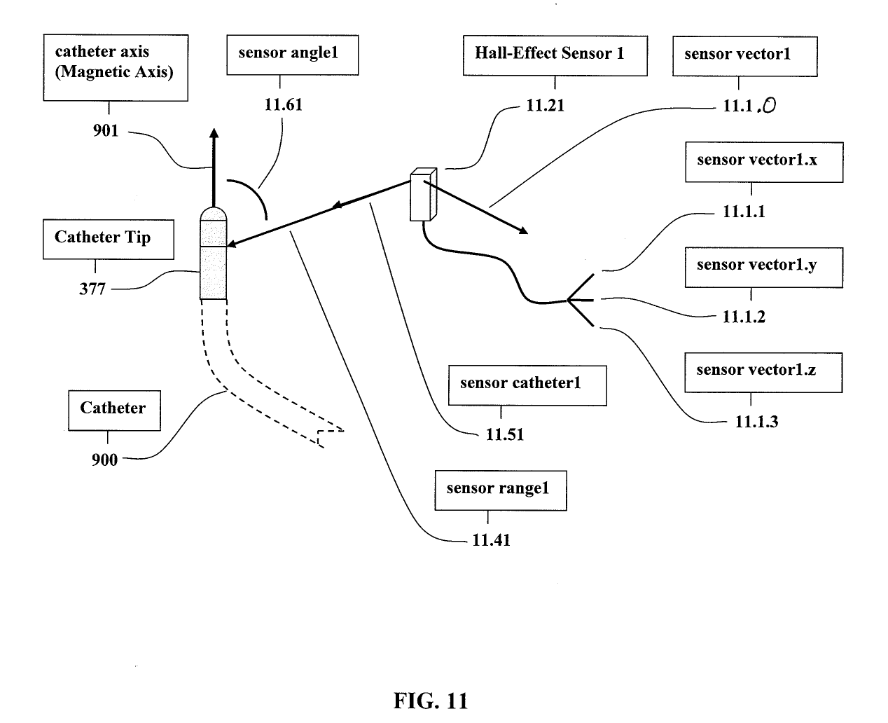

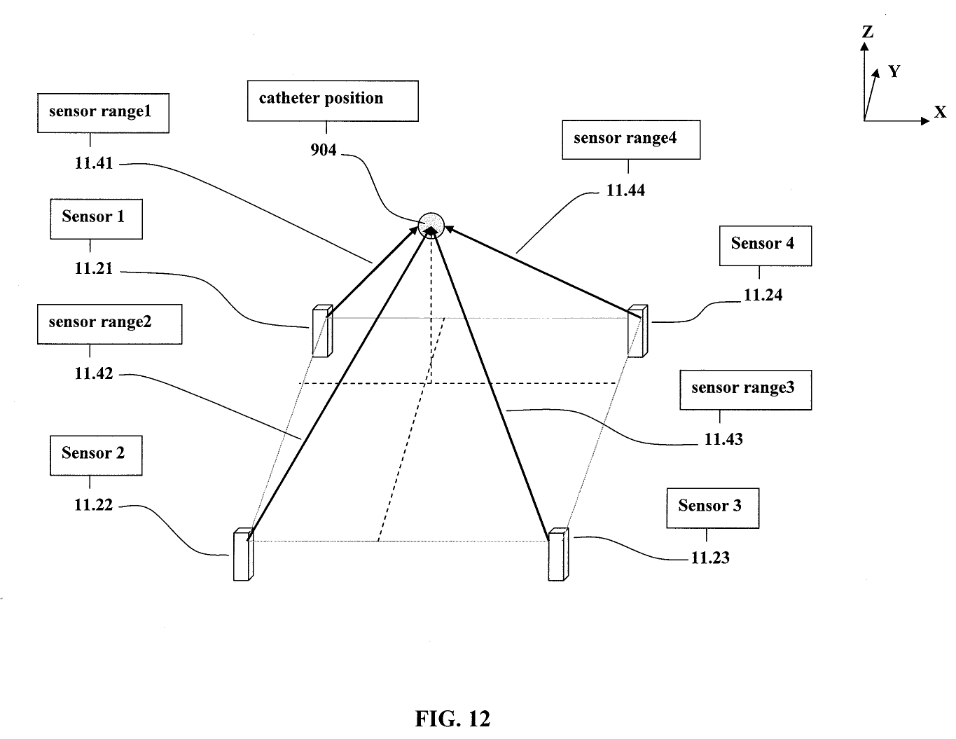

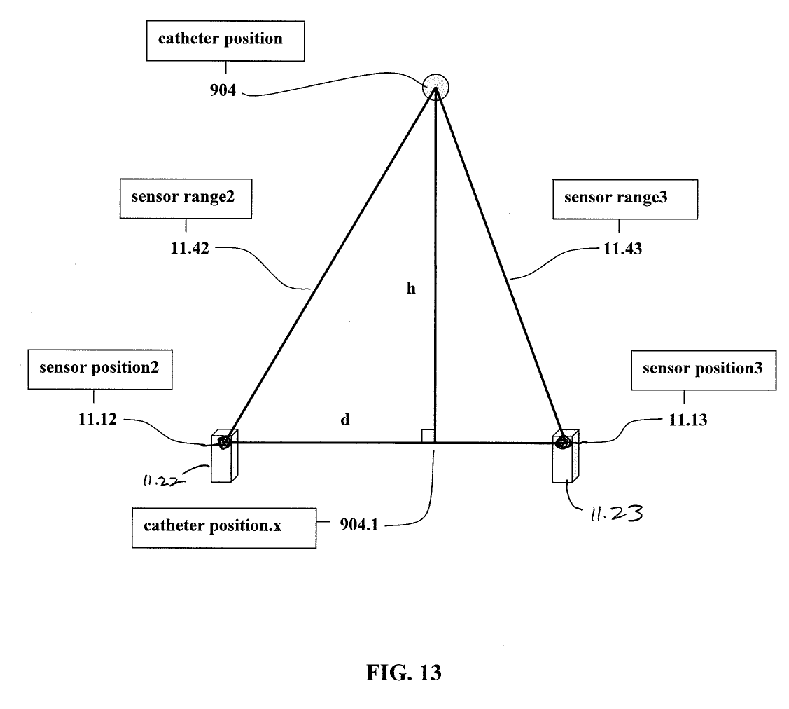

FIG. 10 is a block diagram showing the CGCI detection unit 11 as a top-level system that is responsible for providing the fiducial alignment system 12 with catheter tip actual position and orientation, AP. The CGCI detection unit 11 includes the hardware apparatus 11.7 and software algorithms 11.8 required to determine the position and orientation, AP, of the magnetic catheter tip 377. Four three-axis magnetic sensors 11.21-11.24 sense the magnetic field around the patient 1, as shown in FIG. 12. The magnetic sensors 11.21-11.24 can be sensing coils, Hall-effect sensors, SQUID sensors, etc. These sensor readings 11.1.0-3, 11.41-11.44 and 11.21-11.24 in FIGS. 11-14 are fed to the detection software algorithm 11.8, contained within the operator console 13. The CGCI controller's 501 own magnetic field is subtracted in calibration and filtering unit 11.6 in FIG. 10, and the remaining field is used to calculate the catheter tip's position and orientation, AP 902. The moving heart can be seen as a static object by using the QRS heartbeat synchronization unit 11.5 in FIG. 10 to limit the position readings to the most stable portion of the heartbeat.

FIG. 10A is a block diagram showing the iterative process used in the catheter detection unit software algorithm 11.8 described herein.

FIG. 11 is a diagram showing the signals and quantities involved in the calculation of catheter tip position and orientation, AP 902. The catheter tip's magnetic axis is identical to the tip's orientation 901. The diagram shows only one sensor 11.21 for simplicity, but it is to be understood that the arrangement is replicated for each of the multiple sensors 11.21-11.24. Each Hall-Effect sensor 11.21-11.24 records a magnetic vector, namely a corresponding sensor vector 11.1-11.4, represented by sensor vector-1 11.1, in FIG. 11 and as viewed from its location, a corresponding sensor position 11.11-11.14. The sensor's position in the magnetic field is determined by its angle, sensor angle 11.61-11.64 (see e.g., FIG. 11 sensor angle 1 11.61) and distance between the sensor and catheter tip 3 77, the sensor range 11.41-11.44 (see e.g., FIG. 11 sensor range-1, 11.41). The actual direction from the sensor to the catheter is the unit vector, sensor catheter 11.51-11.54 represented in FIG. 11 by sensor catheter-1 11.51.

FIG. 12 is a geometric representation of the method by which the catheter position 904 is determined by triangulation using the sensor range1 11.41, sensor range2 11.42, sensor range3 11.43, and sensor range4 11.44. The above sensor range values are generated by using sensors1-4, 11.21, 11.22, 11.23, 11.24, respectively.

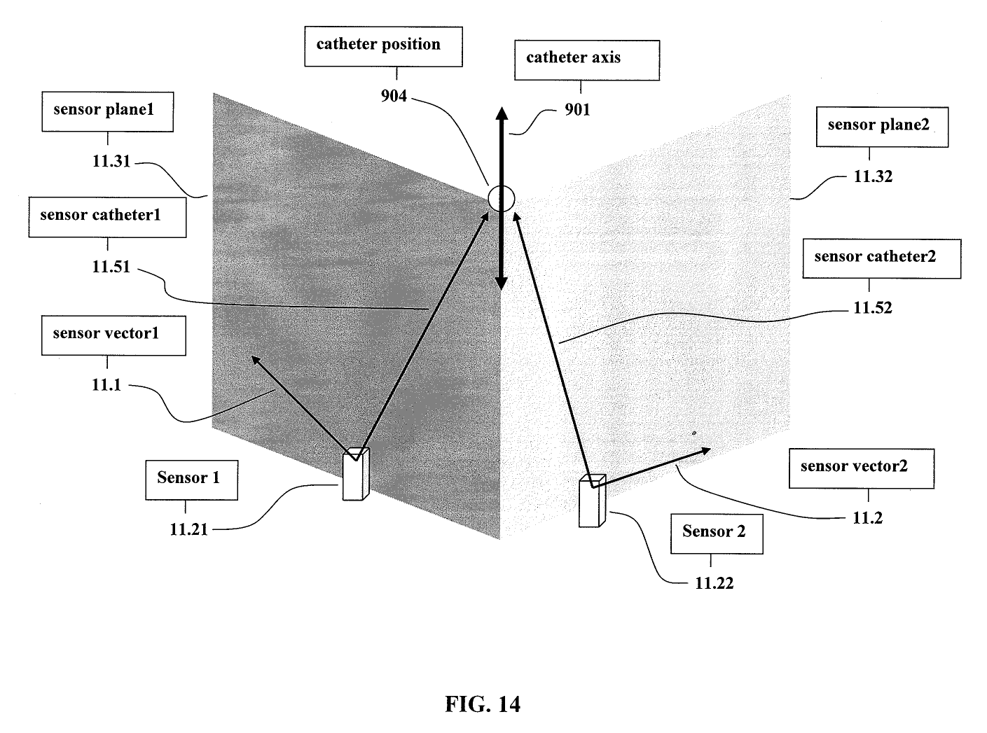

FIG. 13 shows how each adjacent sensor pair is used to triangulate either catheter position.x or catheter position.y. FIG. 13 specifically depicts the scheme whereby sensor-2 11.22 and sensor-3 11.23 are juxtapositioned to locate the x-coordinate of catheter position 904, resulting in catheter position.x 904.1. Sensor vector values 11.1-11.4 are converted to sensor range values 11.41-11.44 by a range equation. The above values are used in triangulation of catheter position 904.

The process of calculating the position of the catheter tip is iterative and a detailed example of such process is described below. The following example demonstrates how the catheter detection algorithm 11.8 in FIG. 10A is used to define the position and orientation of the catheter tip 377 in global CGCI coordinates 100. The process of calculating the position and orientation of the catheter tip shown below involves three distinct stages: calculating the range, calculating the position and calculating the orientation of the catheter tip 377.

The range equation below converts the magnitude of the magnetic sensor reading sensor vectors 11.1-11.4 to a distance from the corresponding sensor, namely sensor ranges 11.41-11.44.

Sensor Range=A·|Sensor Vector|^{1/3}+B

Where the general forms of A and B are:
A=f₁(Sensor Angle)=a·SensorAngle+b
B=f₂(Sensor Angle)=a·sine (b·SensorAngle+c)+d
Sensor Angle=arc cos [Catheter Axis(Catheter Position−Sensor Position)]
   where Catheter Axis, Catheter Position and Sensor Position are vectors of the respective catheter axis, catheter position and sensor positions, is the vector dot product, and f₁ and f₂ are magnetic field geometry functions who's coefficients a,b,c, and d are calculated and/or determined experimentally for each catheter type.

For Example, a 12-F catheter tip's magnetic field has been characterized over the expected sensor operating range and found to have the field shape described by the equations below:

A=64.0−7.0·Sensor Angle

B=2.35·sin (2.45·Sensor Angle+1.35)+0.05

The specific numbers provide in the present example, and in the examples that follow pare provided by way of example and are not limiting to the claims.

The range equation is thus:

Sensor Range=(64−7.0·Sensor Angle)·|Sensor Vector|^{-1/3}+2.35·sin (2.45·Sensor Angle+1.35)+0.05

The range finder triangulation takes the sensor range values 11.41-11.44 and triangulates catheter position 904. In FIG. 12 as an example the x-coordinate of the catheter tip, Catheter Position.x, is determined by sensor pair 1 and 4 and sensor pair 2 and 3. The y-coordinate, Catheter Position.y, is determined by sensor pair 1 and 2 and sensor pair 3 and 4. The z-coordinate, Catheter Position.z, is an average of four solutions to the standard triangle equation.

The sensor range is first calculated for an average sensor angle value from the Range Equation (1). The exact value is not important, as it is just a starting point for the iterative solution. Assume, for example, that the sensor range values for a 12F catheter tip 377 in a given position are:

Sensor Range1=45·|Sensor Vector1|^{-1/3}+0.184

Sensor Range2=45·|Sensor Vector2|^{-1/3}0.184

Sensor Range3=45·|Sensor Vector3|^{-1/3}0.184

Sensor Range4=45·|Sensor Vector4|^{-1/3}0.184

Heron's formula is used to find the Catheter Position.y between sensor pair 1 and 2 and sensor pair 3 and 4. The Catheter Position.x is the average solution between sensor pair 2 and 3 and sensor pair 1 and 4.

For sensors 1 and 2, determine a solution for Catheter Position.y:

S=(Sensor Range1+Sensor Range2+|Sensor Position1−Sensor Position2|)/2

K=(S·(S−Sensor Range1)·(S−Sensor Range2)·(S−|Sensor Position1−Sensor Position2|))^{1/2}

h=2 K /(|Sensor Position1−Sensor Position2|)

d12=(Sensor Range1²=h²)^{1/2}

Catheter Position.y=Sensor Position1.y+/−d12

For sensors 3 and 4, determine a second solution for Catheter Position.y:

S=(Sensor Range3+Sensor Range4+|Sensor Position4−Sensor Position3|)/2

K=(S·(S−sensor range3)·(S−Sensor Range4)·

(S−|Sensor Position4−Sensor Position3|))^{1/2}

h=2 K/(Sensor Position4−Sensor Position3|)

d34=(Sensor Range4²−h²)^{1/2}

Catheter Position.y=Sensor Position4.y+/−d34

Similarly for sensors 1 and 4, determine a solution for Catheter Position.x:

S=(Sensor Range1+Sensor Range4+|Sensor Position4−Sensor Position1|)/2

K=(S·(S−Sensor Range1)·(S−Sensor Range4)·

(S−|Sensor Position4−Sensor Position1|))^{1/2}

h=2 K /(Sensor Position4−Sensor Position1|)

d14=(Sensor Range4²−h²)^{1/2}

Catheter Position.x=Sensor Position4.x+/−d14

For sensors 2 and 3, can determine a second solution for Catheter Position.x:

S=(Sensor Range2+Sensor Range3+|Sensor Position3−Sensor Position2|)/2

K=(S·(S−Sensor Range2)·(S−Sensor Range3)·(S|Sensor Position3−Sensor Position2|))^{1/2}

h=2 K /(|Sensor Position3−Sensor Position2|)

d23=(Sensor Range3²−h²)^{1/2}

Catheter Position.x=Sensor Position3.x+/−d23

The solutions are then averaged to find Catheter Position.x and Catheter Position.y.

The z-coordinate of the catheter position, Catheter Position.z, is the average of the four solutions to the simple right-triangle equation.

Catheter Position.z=(Sensor Range1²−Catheter Position.x²−Catheter Position.y²)^{1/2}

Catheter Position.z=(Sensor Range2²−Catheter Position.x²−Catheter Position.y²)^{1/2}

Catheter Position.z=(Sensor Range3²−Catheter Position.x²−Catheter Position.y²)^{1/2}

Catheter Position z=(Sensor Range4²−Catheter Position.x²−Catheter Position.y²)^{1/2}

Now that the catheter position 904 in the x, y, and z coordinates, are known, one can determine the catheter's orientation, catheter axis 901.

FIG. 14 illustrates the algorithm for finding the catheter tip's magnetic axis or catheter axis 901, using the intersection of magnetic sensor planes. Each sensor's magnetic vector, sensor vectors 11.1-11.4, is assumed to be coplanar with the catheter tip's magnetic axis, catheter axis 901. Given the position of the catheter tip or catheter position 904, the sensor-to-catheter tip vector, represented by sensor catheter vectors 11.51 and 11.52 in Fig. 14, is calculated and crossed with each sensor's magnetic field reading, namely the corresponding sensor vector 11.1-11.4, to get the normal vector that defines the corresponding magnetic plane, sensor plane, which are represented in FIG. 14 by sensor planes 11.31 and 11.32.

First, the unit vector from Sensor Position and Catheter Position is calculated as follows:

$$Sensor Catheter 1 = \frac{( Catheter Position - Sensor Position 1 )}{\vert ( Catheter Position - Sensor Position 1 ) \vert}$$

$$Sensor Catheter 2 = \frac{( Catheter Position - Sensor Position 2 )}{\vert ( Catheter Position - Sensor Position 2 ) \vert}$$

$$Sensor Catheter 3 = \frac{( Catheter Position - Sensor Position 3 )}{\vert ( Catheter Position - Sensor Position 3 ) \vert}$$

$$Sensor Catheter 4 = \frac{( Catheter Position - Sensor Position 4 )}{\vert ( Catheter Position - Sensor Position 4 ) \vert}$$

The sensor catheter vectors are then crossed with the magnetic sensor readings to get the plane normal vectors as follows:

Sensor Plane1=Sensor Vector1×Sensor Catheter1

Sensor Plane2=Sensor Vector2×Sensor Catheter2

Sensor Plane3=Sensor Vector3×Sensor Catheter3

Sensor Plane4=Sensor Vector4×Sensor Catheter4

The intersection of two planes is a line defined by the vector cross-product of the two plane's normal vectors. Only planes from adjacent sensors are crossed. If the catheter axis 901 which is found as the solution is not in the same plane as the normal vector with the sensor catheter 11.5x vector, the solution is reversed to the opposite direction.

Catheter Axis=Sensor Plane1×Sensor Plane2

Catheter Axis=Sensor Plane4×Sensor Plane3

Catheter Axis=Sensor Plane1×Sensor Plane4

Catheter Axis=Sensor Plane3×Sensor Plane2

The catheter axis values are checked for proper sign, and the four solutions are averaged to give catheter axis 901.

Catheter Axis is now used to recalculate the distances from each sensor to the catheter tip.

Sensor Angle1=arc cos(Catheter AxisSensor Catheter1)radians

Sensor Angle2=arc cos(Catheter AxisSensor Catheter2)radians

Sensor Angle3=arc cos(Catheter AxisSensor Catheter3)radians

Sensor Angle4=arc cos(Catheter AxisSensor Catheter4)radians

The sensor range values are updated based on the new sensor angle values. In the present example, the updated equations with the 12F catheter example values assumed above are:

Sensor Range1=(64-7·Sensor Angle1)·|Sensor Vector1|^{-1/3}+2.35·Sin(2.45·Sensor Angle1+1.35)+0.05

Sensor Range2=(64-7·Sensor Angle2)·|Sensor Vector2|^{-1/3}+2.35·Sin(2.45·Sensor Angle2+1.35)+0.05

Sensor Range3=(64-7·Sensor Angle3)·|Sensor Vector3|^{-1/3}+2.35·Sin(2.45·Sensor Angle3+1.35)+0.05

Sensor Range4=(64-7·Sensor Angle4)·|Sensor Vector4|^{-1/3}+2.35·Sin(2.45·Sensor Angle4+1.35)+0.05

The catheter position is retriangulated from these new values, and the loop repeats in reiterative cycles until the desired accuracy is found by comparison between successive values.

Thus, the process for determining the position and orientation of the catheter includes::
1) Approximate the catheter tip magnetic field shape (e.g., as a sphere or non-spherical shape).
2) Calculate initial values for the sensor range from each sensor to the catheter tip.
3) Triangulate the position of the catheter tip or catheter position.
4) Solve for the magnetic axis, catheter axis based on catheter position.
5) Modify the sensor range values using catheter axis.
6) Re-calculate the catheter position.
7) Return to step 4, until sufficient accuracy has been reached.

Using the technique for determining position and orientation of the catheter tip, as noted above, as a further illustration and example, find catheter position 904 and catheter axis 901 for a catheter tip placed in the tricuspid valve. The only known values are the sensor positions 11.1x, their three dimensional magnetic field readings 11.x and the magnetic characteristics of the catheter tip.

With the given sensor data and using the method described above, the catheter tip's position and axis is found at

Catheter Position=(−11.7-14.6 2.1)

Catheter Axis=<−0.408 0.092 −0.908>

The four three dimensional Hall-effect sensors are assumed in this example to be located at coordinates:

Sensor Position1=(−50 50 −150)

Sensor Position2=(−50 −50 −150)

Sensor Position3=(50 −50 −150)

Sensor Position4=(50 50 −150)

The four sensor readings are given as “magnitude x<unit vector>”

Sensor Vector1=0.0187 x<−0.017 0.625 −0.780>

Sensor Vector2=0.0222 X<−0.038 −0.504 −0.863>

Sensor Vector3=0.0240 X<0.888 −0.368 −0.275>

Sensor Vector4=0.0205 X<0.880 0.402 −0.254 >

First, the effective distance to each sensor is calculated using the magnitude of the magnetic signal that it is receiving. Since the orientation of the sensor to the magnetic field in unknown, it is possible to assume a spherical field for the first iteration in this example. It must be understood, however, that this assumption is not a limitation and non-spherical or other field patterns could be accommodated, including empirically-determined arbitrary field patterns.

Sensor Range1=45·|Sensor Vector1|^{-0.333}+0.185

Sensor Range1=169.5 mm

Sensor Range2=45·|Sensor Vector2|^{-0.333}+0.185

Sensor Range2=160.1 mm

Sensor Range3=45·|Sensor Vector3|−0.333+0.185

Sensor Range3=156.0 mm

Sensor Range4=45·|Sensor Vector4|^{-0.333}+0.185

Sensor Range4=164.4 mm

Using Heron's or Hero's triangle formula for each pair of adjacent range values, two solutions are found for Catheter Position.x and Catheter Position.y. For Sensor Range1, Sensor Range2 and the distance between the sensors of 100 mm, the y-coordinate of catheter position, Catheter Position.y is:

S=(Sensor Range1+Sensor Range2+100 mm)/2

K=(S·(S−Sensor Range1)·(S−Sensor Range2)·(S−100 mm))^{1/2}

h=2 K/100 mm

Catheter Position.y=50−(Sensor Range1²−h²)^{1/2}

Catheter Position.y=−15.5 mm

Repeating for sensors 3 and 4 gives:

S=(Sensor Range3+Sensor Range4+100 mm)/2

K=(S·(S−Sensor Range3)·(S−Sensor Range4)·(S−100 mm))^{1/2}

h=2K/100 mm

Catheter Position.y=50−(Sensor Range4²−h²)^{1/2}

Catheter Position.y=−13.5 mm

For the catheter's x-coordinate, use sensors 1 and 4

S=(Sensor Range1+Sensor Range4+100 mm)/2

K=(S·(S−Sensor Range1)·(S−Sensor Range4)·(S−100 mm))^{1/2}

h=2K/100 mm

Catheter Position.y=50−(Sensor Range4²−h²)/

Catheter Position.x=8.5 mm

And for sensors 2 and 3:

S=(Sensor Range2+Sensor Range3+100 mm)/2

K=(S·(S−Sensor Range2)·(S−Sensor Range3)·(S−100 mm))^{1/2}

h=2K/100 mm

Catheter Position.y=50−(Sensor Range3²−h²)^{1/2}

Catheter Position.x=6.5 mm

This gives an average Catheter Position.y=−14.5 mm and Catheter Position.x=7.5 mm.
To find Catheter Position.z, the Pythagorean theorem is used:

Catheter Position.z=(Sensor Range1²−(−50−CatheterTip.x)²−(50−CatheterTip.y)²)^{1/2}−150

Catheter Position.z=−4.2 mm

The initial value for catheter position for the first iteration is significantly inaccurate. Catheter Position =(7.5, −14.5, −4.2) while in this example the true position is (−11.7, −14.6, 2.1)

Catheter position 904 is now used to determine the magnetic axis, catheter axis 901, using the intersecting plane method. Each plane is defined by its normal vector, sensor plane, which is the cross-product of sensor vector and the vector from the sensor to catheter position, sensor catheter.

For the sensor-to-catheter tip unit vectors:

$$Sensor Catheter 1 = \frac{( Catheter Position - Sensor Position 1 )}{\vert ( Catheter Position - Sensor Position 1 ) \vert}$$

$$Sensor Catheter 1 = < 0.339 - 0.381 \; 0.86 > \\ Sensor Catheter 2 = \frac{( Catheter Position - Sensor Position 2 )}{\vert ( Catheter Position - Sensor Position 2 ) \vert}$$

$$Sensor Catheter 2 = < 0.358 \; 0.221 \; 0.907 > \\ Sensor Catheter 3 = \frac{( Catheter Position - Sensor Position 3 )}{\vert ( Catheter Position - Sensor Position 3 ) \vert}$$

$$Sensor Catheter 3 = < - 0.272 \; 0.228 \; 0.935 > \\ Sensor Catheter 4 = \frac{( Catheter Position - Sensor Position 4 )}{\vert ( Catheter Position - Sensor Position 4 ) \vert}$$

$$Sensor Catheter 4 = < - 0.258 - 0.391 \; 0.884 >$$

The sensor plane normal unit vectors are:

Sensor Plane1=Sensor Vector1×Sensor Catheter/|Sensor Vector1|

Sensor Plane1=<0.241 −0.250 −0.206>

Sensor Plane2=Sensor Vector2×Sensor Catheter/|Sensor Vector2|

Sensor Plane2=<−0.267 −0.274 0.172>

Sensor Plane3=Sensor Vector3×Sensor Catheter/|Sensor Vector3|

Sensor Plane3=<−0.281 −0.755 0.102>

Sensor Plane4=Sensor Vector4×Sensor Catheter/|Sensor Vector4|

Sensor Plane4=<0.256 −0.712 −0.240 >

The four solutions for catheter axis are:

Catheter Axis=Sensor Plane1×Sensor Plane2=<−0.597 0.081 −0.798>

Catheter Axis=Sensor Plane4×Sensor Plane3=<−0.540 0.088 −0.837>

Catheter Axis=Sensor Plane1×Sensor Plane4=<−0.625 0.038 −0.779>

Catheter Axis=Sensor Plane3×Sensor Plane2=<−0.629 0.131 −0.766 >

The average of the four solutions is:

Catheter Axis=<−0.598 0.085 −0.795>

The average is thus an approximation for the known value catheter axis =<−0.408 0.092−0.908 >

The new value for Catheter Axis is now used to correct the calculations for the effective radius, since they are dependent on the orientation of the sensors in the catheter tip's magnetic field. The angle of the sensors' positions to the magnetic axis are:

Sensor Angle1=Arc Cos(Catheter Axis·Sensor Catheter1)

Sensor Angle1=156.8

Sensor Angle2=Arc Cos(Catheter Axis·Sensor Catheter2)

Sensor Angle2=156.5

Sensor Angle3=Arc Cos(Catheter Axis·Sensor Catheter3)

Sensor Angle3=124.1

Sensor Angle4=Arc Cos(Catheter Axis·Sensor Catheter4)

Sensor Angle4=125.6

Sensor Range1=(64-7·Sensor Angle1)·|Sensor Vector1|−0.333+2.35·sin(2.45·Sensor Angle1+1.35)+0.05

Sensor Range2=(64-7·Sensor Angle2)·|Sensor Vector2|−0.333+2.35 sin(2.45·Sensor Angle2+1.35)+0.05

Sensor Range3=(64-7·Sensor Angle3)·|Sensor Vector3|−0.333+2.35 sin(2.45·Sensor Angle3+1.35)+0.05

Sensor Range4=(64-7·Sensor Angle4)·|Sensor Vector4 −0.333+2.35·sin(2.45·Sensor Angle4+1.35)+0.05

Sensor Range1=171.1 mm

Sensor Range2=161.9 mm

Sensor Range3=170.0 mm

Sensor Range4=178.6 mm

Using these new values in Heron's formula, compute the solutions for the x and y coordinate of catheter position.

Catheter Position.y=−15.3 mm

Catheter Position.y=−15.0 mm

Catheter Position.x=−13.1 mm

Catheter Position.x=−13.5 mm

Averaging these and solving for catheter position.z give the first iteration values

Catheter Position=(−13.3 −15.2 3.8)

Catheter Axis=<−0.60 0.084 −0.795 >

The second iteration is performed by plugging CatheterPostion back into the equations to recalculate sensor catheter, sensor plane and catheter axis. The sensor range values are recalculated and the new catheter position is found.

Catheter Position=(−11.5 −14.34 1.54)

Catheter Axis=<−0.386 0.096 −0.917 >

The third iteration in the same manner gives the values:

Catheter Position=(−11.9 −14.54 1.91)

Catheter Axis=<−0.412 0.090 −0.907 >

The results from the third iteration compare favorably with the actual values:

Catheter Position=(−11.7 −14.6 2.1)

Catheter Axis=<−0.408 0.092 −0.908 >

Further iterations would give greater accuracy, if required.

Using the method for determining position and orientation of the catheter tip described above, as a further example, determine catheter position 904 and catheter axis 901 for a catheter tip placed in the top right pulmonary vein. The only known values are the sensor positions, their three dimensional magnetic field readings and the magnetic characteristics of the catheter tip.

With the given sensor data, the catheter tip's position and axis should be found at

Catheter Position=(61.1 −1.1 −14.6)

Catheter Axis=<−0.916 0.090 −0.391 >

The four three dimensional Hall-effect sensors are assumed to be located at the coordinates:

Sensor Position1=(−50 50 −150)

Sensor Position2=(−50 −50 −150)

Sensor Position3=(50 −50 −150)

Sensor Position4=(50 50−150)

The four sensor readings are given in this example as “magnitude x<unit vector>”: Sensor Vectorl =0.0162 X<-0.147 0.399-0.905>Sensor Vector2=0.0170 X<-0.094-0.535-0.840>Sensor Vector3=0.0416 X<0.853-0.365-0.372>Sensor Vector4=0.0402 X<0.845 0.238-0.479 >

First, the effective distance to each sensor is calculated based on the magnitude of the magnetic signal that it is receiving. Since the orientation of the sensor to the magnetic field in unknown, a spherical field is assumed for the first iteration. Again this assumption need not be made and arbitrary field patterns can be accommodated by appropriate transformation functions.

Sensor Range1=45·|Sensor Vector1|^{-0.333}+0.185

Sensor Range1=177.8 mm

Sensor Range2=45·|Sensor Vector2|^{-0.333+0.185}

Sensor Range2=175.0 mm

Sensor Range3=45·|Sensor Vector3|^{-0.333}+0.185

Sensor Range3=129.9 mm

Sensor Range4=45·|Sensor Vector4^{-0.333}+0.185

Sensor Range4=131.4 mm

Using Heron's triangle formula for each pair of adjacent range values, two solutions are found for Catheter Position.x and Catheter Position.y. For Sensor Range1, Sensor Range2 and the distance between sensors of 100 mm, the y-coordinate of catheter position, is:

S=(Sensor Range1+Sensor Range2+100 mm)/2

K=(S·(S−Sensor Range1)·(S−Sensor Range2)·(S−100 mm))^{1/2}

h=2K/100 mm

Catheter Position.y=50−(Sensor Rangel²- h²)^{1/2}
Catheter Position.y=−5.0 mm

Repeating for Sensors 3 and 4 gives:

S=(Sensor Range3+Sensor Range4+100 mm)/2

K=(S·(S−Sensor Range3)·(S−Sensor Range4) ·(S−100 mm))^{1/2}

h=2K/100 mm

Catheter Position.y=50−(Sensor Range4²−h²)^{1/2}

Catheter Position.y=−1.9 mm

For the Catheter's x-coordinate, use sensors 1 and 4:

S=(Sensor Range1+Sensor Range4+100 mm)/2

K=(S·(S−Sensor Range1) · (S−Sensor Range4)·(S−100 mm))/2

h=2K/100 mm

Catheter Position.y=50+(Sensor Range4²−h²)^{1/2}

Catheter Position.x=71.7 mm

And for sensors 2 and 3:

S=(Sensor Range2+Sensor Range3+100 mm)/2

K=(S·(S−Sensor Range2)·(S−Sensor Range3)·(S−100 mm))^{1/2}

h=2K/100 mm

Catheter Position.y=50+(Sensor Range3²−h²) 2

Catheter Position.x=69.7 mm

This gives an average Catheter Position.y=−3.5 mm and Catheter Position.x=70.2 mm

To find Catheter Position.z, the Pythagorean theorem is used:

Catheter Position.z=(Sensor Rangel²−(−50−CatheterTip.x)²−(50−CatheterTip.y)²)^{1/2}−150

Catheter Position.z=−30.4 mm

The initial value for Catheter Position for iteration 1 is significantly inaccurate, Catheter Position =(69.7 −3.5 −30.4) where the true position =(61.1-1.1-14.6)

Catheter position is now used to determine the magnetic axis, catheter axis, using the intersecting plane method as described above. Each plane is defined by its normal vector, sensor plane, which is the cross-product of sensor vector and the vector from the sensor to Catheter Position, sensor catheter. For the sensor-to-catheter tip unit vectors:

$$Sensor Catheter 1 = \frac{( Catheter Position - Sensor Position 1 )}{\vert ( Catheter Position - Sensor Position 1 ) \vert}$$

$$Sensor Catheter 1 = < 0.676 - 0.301 \; 0.673 > \\ Sensor Catheter 2 = \frac{( Catheter Position - Sensor Position 2 )}{\vert ( Catheter Position - Sensor Position 2 ) \vert}$$

$$Sensor Catheter 2 = < 0.684 \; 0.264 \; 0.680 > \\ Sensor Catheter 3 = \frac{( Catheter Position - Sensor Position 3 )}{\vert ( Catheter Position - Sensor Position 3 ) \vert}$$

$$Sensor Catheter 3 = < 0.156 \; 0.358 \; 0.921 > \\ Sensor Catheter 4 = \frac{( Catheter Position - Sensor Position 4 )}{\vert ( Catheter Position - Sensor Position 4 ) \vert}$$

$$Sensor Catheter 4 = < 0.152 - 0.404 \; 0.902 >$$

The Sensor Plane normal unit vectors are:

Sensor Plane1=Sensor Vector1×Sensor Catheter/|Sensor Vector1|

Sensor Plane1=<−0.004 −0.513 −0.226>

Sensor Plane2=Sensor Vector2×Sensor Catheter/|Sensor Vector2|

Sensor Plane2=<−0.142 −0.510 0.341>

Sensor Plane3=Sensor Vector3×Sensor Catheter/|Sensor Vector3

Sensor Plane3=<−0.203 −0.843 0.362>

Sensor Plane4=Sensor Vector4×Sensor Catheter/|Sensor Vector4

Sensor Plane4=<0.021 −0.835 −0.377 >

The four solutions for Catheter Axis are:

Catheter Axis=Sensor Plane1×Sensor Plane2=<−0.966, 0.111, −0.235>

Catheter Axis =Sensor Plane4×Sensor Plane3=<−0.952, 0.106, −0.288>

Catheter Axis=Sensor Plane1×Sensor Plane4 =<0.313, −0.384, 0.868>

Catheter Axis=Sensor Plane3×Sensor Plane2=<−0.974, 0.169, −0.152>

The value above for Sensor Plane1×Sensor Plane4 can be excluded by error checking.

The average of the four solutions is

Catheter Axis=<−0.644 0.000 0.048

This is a starting approximation for the known value: Catheter Axis =<−0.916 0.090-0.391 >

The new value for Catheter Axis is now used to correct the calculations for effective radius, since they are dependent on the orientation of the sensors in the catheter tip's magnetic field.

The angle of the sensors' positions to the magnetic axis are

Sensor Angle1=arc cos(Catheter Axis·Sensor Catheter1)

Sensor Angle2=arc cos(Catheter Axis·Sensor Catheter2)

Sensor Angle3=arc cos(Catheter Axis·Sensor Catheter3)

Sensor Angle4=arc cos(Catheter Axis·Sensor Catheter4)

Putting in numerical values from the present example gives:

Sensor Angle 1=113.8

Sensor Angle 2=114.1

Sensor Angle3=93.2

Sensor Angle4=93.1

### Then

$$Sensor Range 1 = ( 64 - 7 \cdot Sensor Angle 1 ) \cdot \vert Sensor Vector 1 \vert - 0.333 + 2.35 \cdot sin ( 2.45 \cdot Sensor Angle 1 + 1.35 ) + 0.05 \\ Sensor Range 2 = ( 64 - 7 \cdot Sensor Angle 2 ) \cdot \vert Sensor Vector 2 \vert - 0.333 + 2.35 \cdot sin ( 2.45 \cdot Sensor Angle 2 + 1.35 ) + 0.05 \\ Sensor Range 3 = ( 64 - 7 \cdot Sensor Angle 3 ) \cdot \vert Sensor Vector 3 \vert - 0.333 + 2.35 \cdot sin ( 2.45 \cdot Sensor Angle 3 + 1.35 ) + 0.05 \\ Sensor Range 4 = ( 64 - 7 \cdot Sensor Angle 4 ) \cdot \vert Sensor Vector 4 \vert - 0.333 + 2.35 \cdot sin ( 2.45 \cdot Sensor Angle 4 + 1.35 ) + 0.05$$

Again putting in numerical values for the present example gives:

Sensor Range1=197.6 mm

Sensor Range2=194.4 mm

Sensor Range3=149.8 mm

Sensor Range4=151.6 mm

Using these new values in Heron's formula, compute the solutions for the x and y coordinates of Catheter Position.

Catheter Position.y=×6.37 mm

Catheter Position.y=×2.64 mm

Catheter Position.x=80.37 mm

Catheter Position.x=76.64 mm

Averaging these and solving for Catheter Position.z give the first iteration values

Catheter Position=(78.5 −4.5 −10.8)

Catheter Axis=<−0.646 0.0 0.049 >

The second iteration is performed by plugging CatheterPostion back into the equations to recalculate Sensor Catheter, Sensor Plane and Catheter Axis. The Sensor Range values are recalculated and the new Catheter Position is found.

Catheter Position=(62.1 −1.65 −15.75)

Catheter Axis=<−0.936 0.086 −0.334>

Similarly the third iteration gives the values:

Catheter Position=(61.4 −1.15 −14.6)

Catheter Axis=<−0.925 0.089 −0.368>

which compare favorably with the actual values:

Catheter Position=(61.1 −1.1 −14.6)

Catheter Axis=<−0.916 0.090 −0.391>

Further iterations would give greater accuracy, if desired.

FIG. 15 is block diagram showing the fiducial alignment system's 12 function in receiving actual catheter position data, AP 902, from the catheter detection unit 11, and aligning it with a moving patient 1. The fiducial alignment system 12 produces the model transformation matrices GP and GO, 12.501 and 12.500 in block 12a that are used to rotate the virtual models 80 with the patient 1 and allows the virtual catheter tip 905 to interact with the moving anatomical models 80, allowing the operator/surgeon to add to and modify the virtual model data 80. The mapping unit 14 and catheter position recording unit 15 use the fiducial alignment system 12 to convert actual catheter position, AP, back to the original virtual models 80. The operation console 13 requires the transformation matrices GP and GO, 12.501 and 12.500 to properly display the virtual models 80.

FIG. 15A are illustrations of the respiration inspiration 1.60.1 and expiration 1.60.2 states shown in front view on the left and side view on the right of the diagram corresponding to the respiration signal 1.60 from the fiducial sensor 12.16 that is used by the respiration compensation algorithm to compensate for the respiration displacement of the heart. The fiducial alignment system 12 separates the cyclic respiration signal 1.60 from the static displacement of the patient 1 and s available medical algorithms to further track organ displacement to accurately position the virtual tip 905 in the desired location.

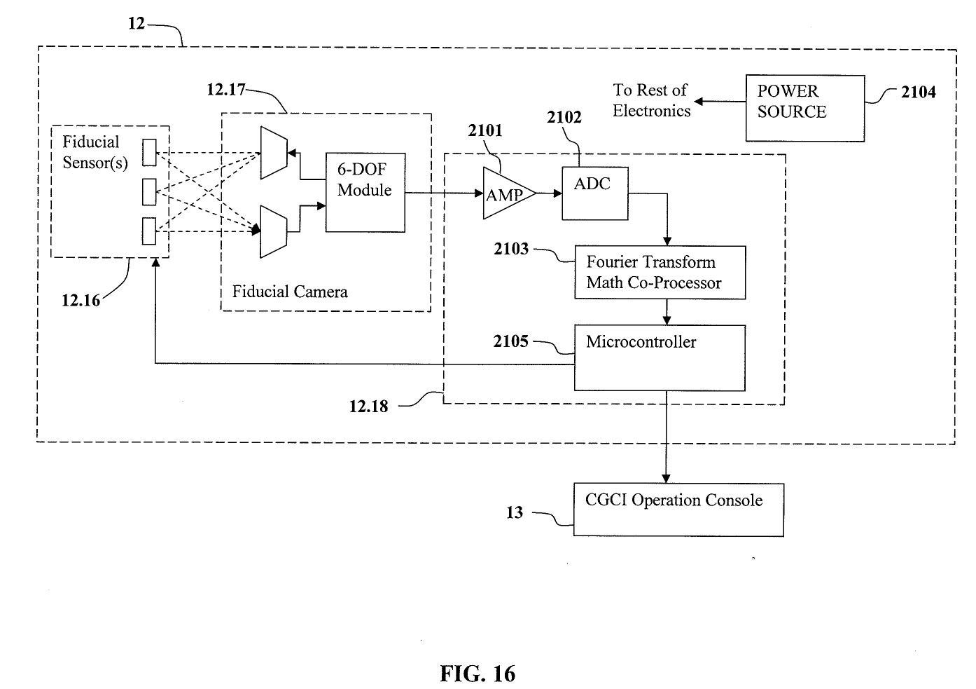

FIG. 16 is a block diagram of the hardware or the CGCI fiducial alignment system 12 and microcontroller 12.18. The fiducial sensor(s) 12.16 are tracked by the fiducial camera 12.17. The fiducial microcontroller and associated hardware 12.18 convert the data from the fiducial camera 12.17 and transmits the fiducial sensor position and orientation to the operation console 13. Imagers 12.17.1 scan the images of fiducial sensors 12.16 to provide spatial and angular data characterizing fiducial sensors 12.16 to logic circuit 12.17.2, which provides analog data signals to amplifier 2101. The output of amplifier 2101 is digitized by ADC 2102 whose output is transformed by Fourier Transform logic circuit 2103. Fourier Transform logic circuit 2103. provides a fiducial data signal representing the spatial and angular position of fiducial sensors 12.16 to microcontroller 2105. The fiducial data is then provided by microcontroller 2105 to console 13.

FIG. 17 is a schematic diagram of the coordinate systems and associated vector used in the fiducial alignment system 12. The CGCI's global coordinate system 100 is converted to the patient's local coordinate system 200 by the relative shift in the fiducial sensor position and orientation 300 from the fiducial sensor fixed position and orientation 400. When the fiducial alignment system 12 is “fixed”, the local coordinate system 200 is given the value of the global coordinate system 100 with a model offset vector MO 405 added. The local position vector (LPV) 406 rigidly links the fiducial sensor position and orientation 300 to the local coordinate system position 204 in FIG. 7. The global orientation transformation matrix 12.500 in FIG. 15 is used to convert the global coordinate axes to the local coordinate axes 201-203.

The CGCI units global coordinate system 100 is defined at the center of the coil array by four vectors, representing the central position within the magnetic coils and each Cartesian axis. In the preferred patient orientation Global axisY is the direction of the patient's head, and Global axisZ is up or perpendicularly directed out of the patient.

In the Global Coordinate System 100

| | |
|---|---|
| Global AxisX | GX(GX.x, GX.y, GX.z) = (1, 0, 0) |
| Global AxisY | GY(GY.x, GY.y, GY.z) = (0, 1, 0) |
| Global AxisZ | GZ(GZ.x, GZ.y, GZ.z) = (0, 0, 1) |
| Global Position | GP(GP.x, GP.y, GP.z) = (0, 0, 0) |

The x, y, and z components of a vector are referred to as sub-assemblies of the vector. GX has components GX.x, GX.y, and GX.z.

The patient's local coordinate system 200, to which all medical data and imagery have been referenced, is defined four similar vectors.

In the Local Coordinate System 200

| | |
|---|---|
| Local AxisX | LX(LX.x, LX.y, LX.z) |
| Local AxisY | LY(LY.x, LY.y, LY.z) |
| Local AxisZ | LZ(LZ.x, LZ.y, LZ.z) |
| Local Position | LP(LP.x, LP.y, LP.z) |

The fiducial system's position and orientation sensor 12.16 is referenced to the global coordinate system 100, and has four similar vectors. These change dynamically as the sensor moves.

Fiducial Sensor Current Position and Orientation 300 are given by:

| | |
|---|---|
| Fiducial AxisX | FX(FX.x, FX.y, FX.z) |
| Fiducial AxisY | FY(FY.x, FY.y, FY.z) |
| Fiducial AxisZ | FZ(FZ.x, FZ.y, FZ.z) |
| Fiducial Position | FP(FP.x, FP.y, FP.z) |

A second set of fiducial coordinates are used as a reference to point where the global coordinate system 100 and local coordinate system 200 have been aligned by a simple position offset vector, MO 405.

The fiducial Sensor position and orientation are given by:

| | |
|---|---|
| Fiducial Fixed AxisX | FFX(FFX.x, FFX.y, FFX.z) |
| Fiducial Fixed AxisY | FFY(FFY.x, FFY.y, FFY.z) |
| Fiducial Fixed AxisZ | FFZ(FFZ.x, FFZ.y, FFZ.z) |
| Fiducial Fixed Position | FFP(FFP.x, FFP.y, FFP.z) |
| Local-Global Model Offset | MO(MO.x, MO.y, MO.z) |
| Local Position Vector | LPV(LPV.x, LPV.y, LPV.z) |

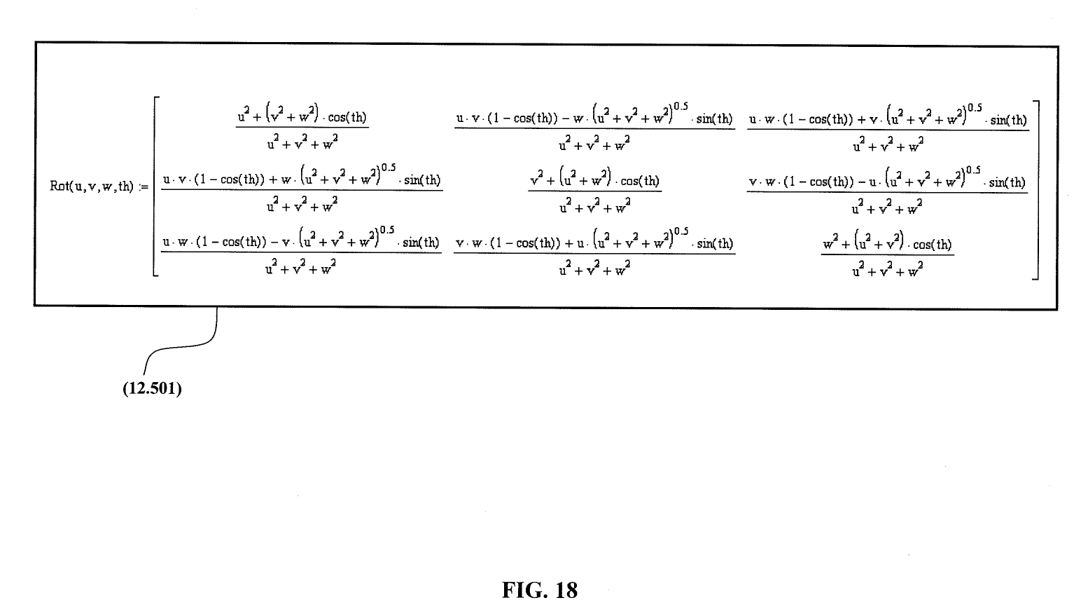

FIG. 18 is the general form of the transformation matrix for rotation about an arbitrary axis (left-handed coordinates), Rot (u,v,w,th) 12.501. Rot (u,v,w,th) 12.501 rotates any vector or point about the vector <u,v,w>by angle th. This is used to rotate the fiducial fixed axes 400 to the current fiducial axes 300 in six standard yaw, pitch, and roll rotations. First, the fiducial fixed orientation is unrotated relative to a global coordinate alignment 100, and then is rotated to the current fiducial sensor orientation 300. The matrix multiplication of these rotations gives the global orientation matrix, GO 12.500. When multiplied by the global orientation matrix, GO 12.500, three dimensional virtual models 80, location points, and vectors are converted from the global coordinate system 100 to the local coordinate system 200.

The global positioning matrix, GP 12.502, is only used to translate three dimensional virtual models 80 from the global position to local position 204. All models and vectors are assumed to be left-handed for three dimensional modeling purposes, and vectors that are right-handed are multiplied by the conversion matrix, LH, before and after transformation.

The global positioning matrix GP is defined from the local position 204 as:

$$GP = [ \begin{matrix}LP . x & 0 & 0 \\ 0 & LP . y & 0 \\ 0 & 0 & LP . z\end{matrix} ]$$

The left-hand to right-hand coordinate conversion matrix, LH, is defined as:

$$LH = [ \begin{matrix}1 & 0 & 0 \\ 0 & 1 & 0 \\ 0 & 0 & - 1\end{matrix} ]$$

Objects with right-handed coordinates, such as position vectors and location points, are multiplied by the right-handed version of GO 12.500, the matrix GOR:

GOR=LH·GO·LH

The fixed reference vectors use include Fiducial Fixed AxisX 401 and its z-component FFX.z 401.3, Fiducial Fixed AxisY 402 and its components FFY.x 402.1, FFY.y 402.2, FFY.z 402.3. The current fiducial vectors use Fiducial AxisX 301 and its z-component FX.z 301.3, Fiducial AxisY 302 and its components FY.x 302.1, FY.y 302.2, FY.z 302.3. Rot[1]=Rotate about axis Fiducial Fixed AxisY by angle arc sin(FFX.z/cos(-arc sin(FFY.z))) Rot[2]=Rotate about axis <FFY.y, -FFY.x, 0>by angle -arc sin(FFY.z) Rot[3]=Rotate about axis Global AxisZ by angle -arc tan(FFY.x/FFY.y) Rot[4]=Rotate about axis Global AxisZ by angle arc tan(FY.x/FY.y) Rot[5]=Rotate about axis <FY.y, -FY.x, 0>by angle arc sin(FY.z ) Rot[6]=Rotate about axis Fiducial AxisY by angle -arc sin(FX.z/cos(arc sin(FY.z)))

GO=Rot[1]·Rot[2]·Rot[3]·Rot[4]Rot[5]·Rot[6]

Using the technique for determining the Global Orientation Matrix GO 12.500 and converting from Global Coordinates 100 to Local Coordinates 200, as noted above, generate GO 12.500 and use it to demonstrate that the Local Catheter Position 914 and Orientation 911 in a rotated patient can be converted back to global Catheter Position 904 and Catheter Axis 901 in an unrotated patient.

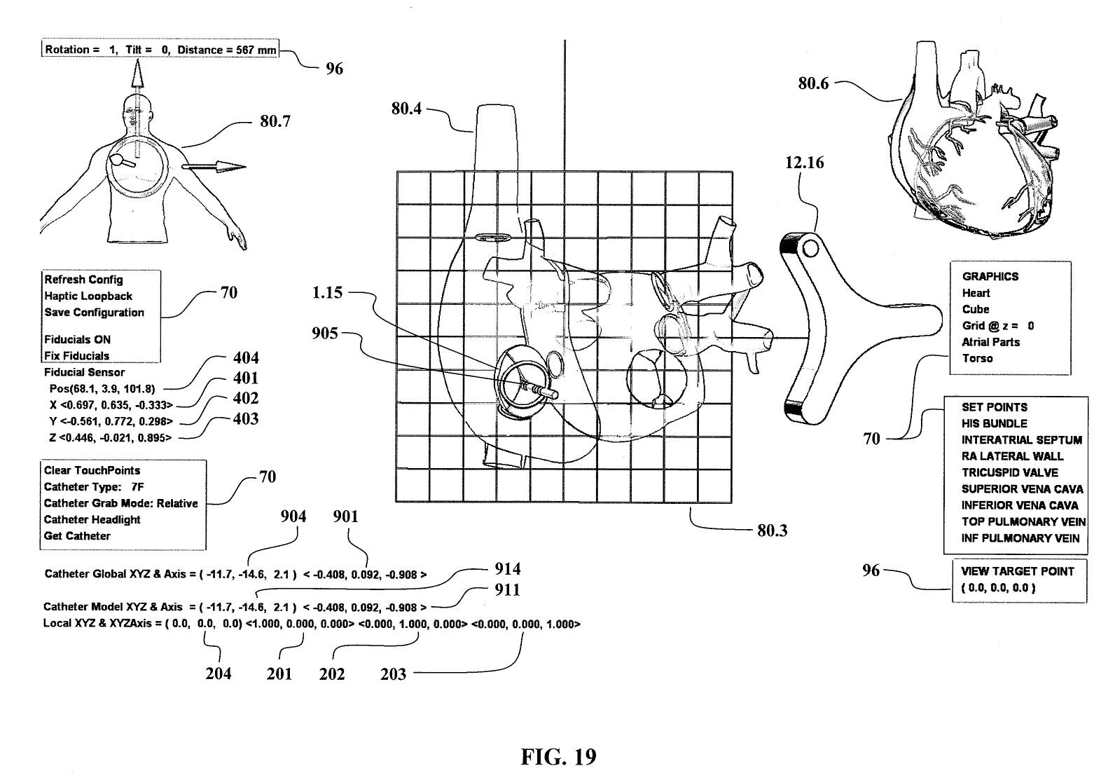

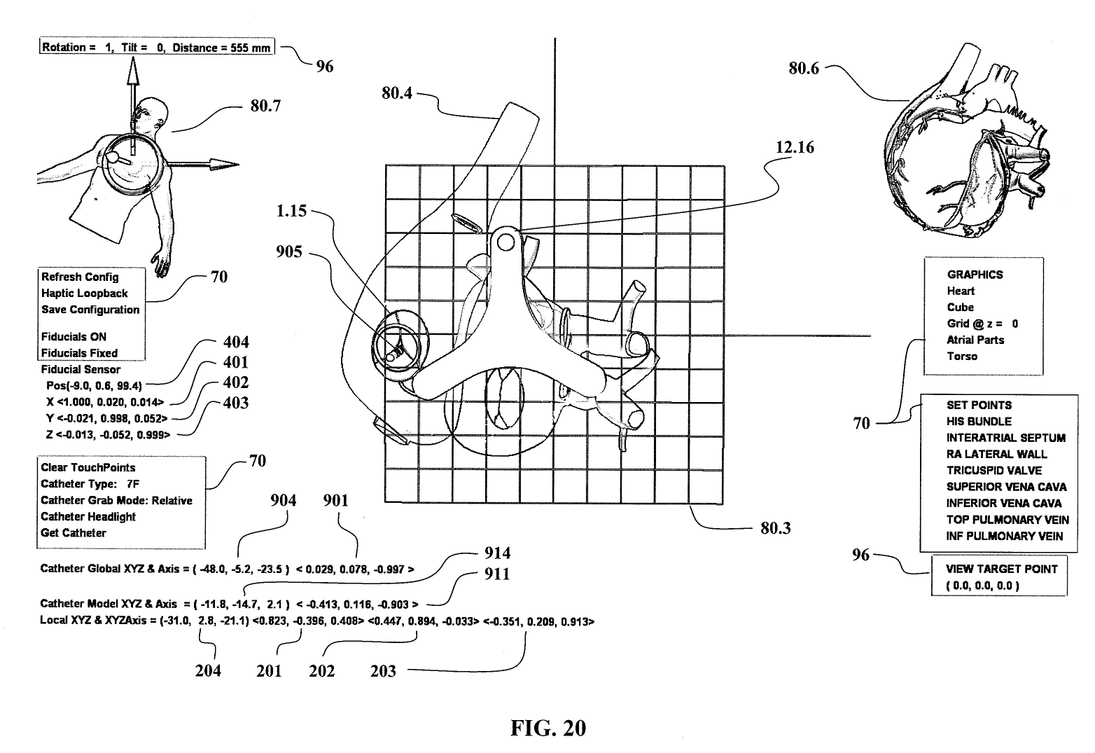

In the screen display of FIG. 19, the virtual catheter tip 905 is hand-placed in the tricuspid valve 1.15 and the fiducial system 12 is fixed. In the screen display of FIG. 20, the patient 1 has been repositioned and the virtual catheter tip 905 is repositioned by hand in the tricuspid valve 1.15. The CGCI system data and results from FIG. 19 and FIG. 20 are:

Fiducial Fixed Position =(68.1, 3.9, 101.8)

Fiducial Fixed Axis X=<0.697, 0.635, -0.333>

Fiducial Fixed Axis Y=<−0.561, 0.772, 0.298>

Fiducial Fixed Axis Z=<0.446, -0.021, 0.895>

Fiducial Position=(−9.0, 0.6, 99.4)

Fiducial Axis X=<1.000, 0.020, 0.014>

Fiducial Axis Y=<−0.021, 0.998, 0.052>

Fiducial Axis Z=<−0.013, −0.052, 0.999>

Catheter in the tricuspid valve, unrotated patient

Catheter Position 1=(−11.7, −14.6, 2.1)

Catheter Axis 1=<−0.408, 0.092, −0.908>

Catheter in tricuspid valve, rotated patient

Catheter Position 2=(−48.0, -5.2, -23.5)

Catheter Axis 2 =<0.029, 0.078, -0.997>

Catheter Model Position 2=(−11.8, -14.7, 2.1)

Catheter Model Axis 2=<−0.413, 0.116, -0.903 >

The rotation matrices, Rot 12.501, are calculated from the system data in the screen displays of FIG. 19 and FIG. 20 and multiplied together to form the global orientation matrix, GO 12.500. The right-handed global orientation matrix, GOR 12.506, is then found by multiplying, before and after, by the right-handed coordinate to left-handed coordinate transformation matrix, LH. Rot[1]=Rotate about the vector <-0.561, 0.772, 0.298>by angle arc sin(−0.333/cos(-arc sin(0.298)))=0.356 rad Rot[2]=Rotate about the vector <0.772, 0.561, 0>by angle -arc sin(0.298)=-0.303 radians Rot[3]=Rotate about the vector <0, 0, 1>by angle -arc tan(−0.561/0.772)=0.628 radians Rot[4]=Rotate about the vector <0, 0, 1>by angle arc tan(−0.021/0.998)=-0.021 radians Rot[5]=Rotate about the vector <0.998, 0.021, 0>by angle arc sin(0.052)=0.052 radians Rot[6]=Rotate about the vector <-0.021, 0.998, 0.052>by angle -arc sin(0.014/cos(arc sin(0.052)))=-0.014 radians GO =Rot(−0.561, 0.772, 0.298, -0.356) Rot(0.772, 0.561, 0, -0.303) Rot(0, 0, 1, 0.628) * Rot(0, 0, 1, -0.021 Rot(0.998, 0.021, 0, 0.052) Rot(−0.021, 0.998, 0.052, -0.014) GOR=LH * GO * LH

To find the local position 204, the initial local position vector, LPV 406, is set to the negative of the fiducial fixed position 404, assuming no initial model offset 405.

LPV 406 is rotated by the right-handed transform, GOR 12.506, and added to the current
fiducial position 304.
Fiducial Fixed Position =(68.1, 3.9, 101.8) mm
Fiducial Position =(−9.0, 0.6, 99.4) mm

LPV=−fiducial fixed position

Local Position =Fiducial Position +LPV·GOR=(−31.12, 2.873, -21.106) mm

The local axes, 201, 202, and 203 are found by multiplying the corresponding global axes by GOR.

Local AxisX=Global AxisX·GOR=<0.823, −0.397, 0.407>

Local AxisY=Global AxisY·GOR=<0.448, 0.894, −0.033>

Local AxisZ=Global AxisZ·GOR=<−0.350, 0.209, 0.913 >

The virtual catheter tip 905 is referenced back to the unrotated model by the matrix inverse of the transformation matrix, GOR, and it is seen to match the initial hand placement.

Catheter Global Axis=(0.029, 0.078, −0.997)

Catheter Local Axis=Catheter Global Axis·GOR⁻¹=(−0.413, 0.116, −0.904)

Given hand-accuracy, the Local Axis in the rotated patient matches the Global Axis in the unrotated patient: <−0.408, 0.092, −0.908 >

The vector from the local position origin 204 to the catheter's global position 904 is rotated back to global coordinates 100 for the catheter's local position 914 with respect to the un-rotated model.

$$Catheterglobal position = ( - 48.0 , - 5.2 , - 23.5 ) mm$$

$$\begin{matrix}Catheterglobal position = ( Catheter global position - \\ local position 204 ) \cdot {GOR}^{- 1} \\ = ( - 11.653 , - 14.694 , 2.037 )\end{matrix}$$

Given hand-accuracy, the local position in the rotated patient matches the global position in the unrotated patient (−11.7, -14.6, 2.1)

Using the technique for determining the global orientation matrix GO and converting from global coordinates 100 to local coordinates 200, as noted above, generate GO and use it to demonstrate that the local Catheter Position 914 and orientation 911 in a rotated patient can be converted back to global Catheter Position 904 and Catheter Axis 901 in an unrotated patient.

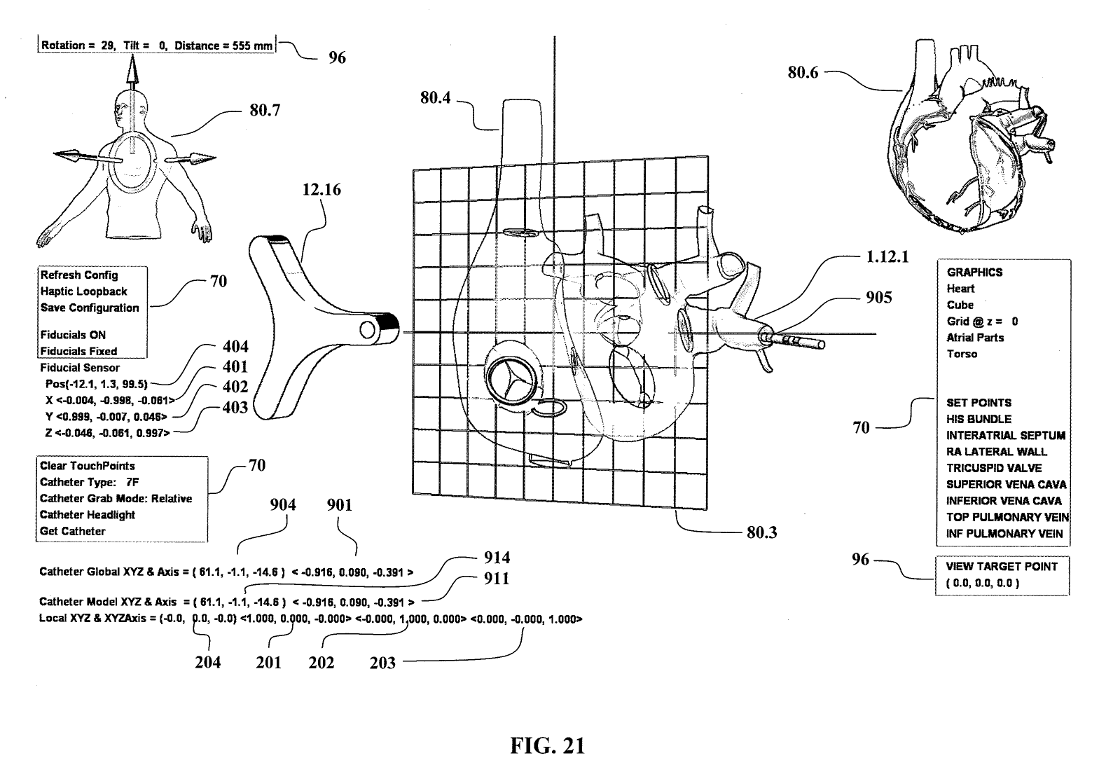

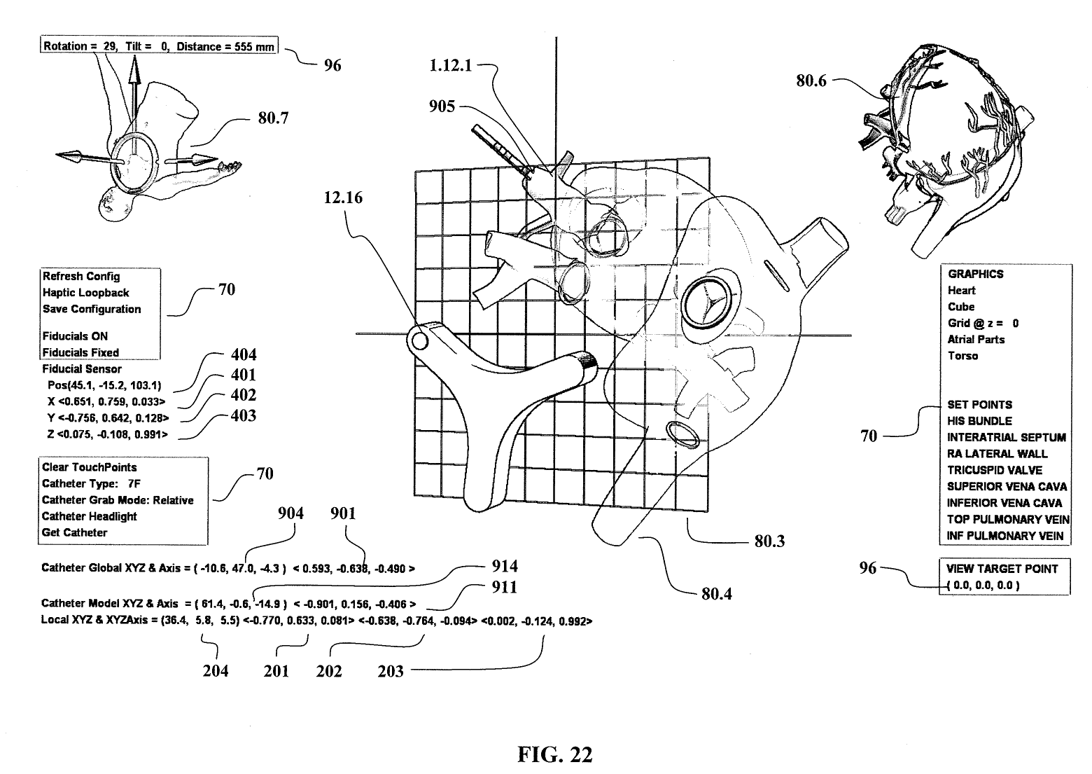

In FIG. 21 as an example, the virtual catheter tip 905 is placed in a pulmonary vein 1.12.1 and the fiducial system 12 is fixed. In FIG. 22, the patient 1 has been repositioned and the catheter tip 905 is repositioned by hand in the same pulmonary vein 1.12.1. The CGCI system data and results from FIG. 21 and FIG. 22 are:

Fiducial Fixed Position =(−12.1, 1.3, 99.5)

Fiducial Fixed Axis X=<−0.004, −0.998, −0.061>

Fiducial Fixed Axis Y=<0.999, −0.007, 0.046>

Fiducial Fixed Axis Z=<−0.046, −0.061, 0.997>

Fiducial Position=(45.1, −15.2, 103.1)

Fiducial Axis X=<0.651, 0.759, 0.033>

Fiducial Axis Y=<−0.756, 0.642, 0.128>

Fiducial Axis Z=<0.075, -0.108, 0.991 >

Catheter in pulmonary vein, unrotated patient

Catheter Position 1=(61.1, −1.1, −14.6)

Catheter Axis 1=<−0.916, 0.090, -0.391>

Catheter in pulmonary vein, rotated patient

Catheter Position 2=(−10.6, 47.0, −4.3)

Catheter Axis 2 =<0.593, −0.638, −0.490>

Catheter Model Position 2=(61.4, −0.6, −14.9)

Catheter Model Axis 2=<−0.901, 0.156, −0.406 >

The rotation matrices, Rot 12.501, are calculated from the system data in FIG. 21 and FIG. 22 and multiplied together to form the global orientation matrix, GO 12.500. The right-handed global orientation matrix, GOR, is then found by multiplying, before and after, by the left-handed to right-handed transformation matrix, LH.

$$\begin{matrix}Rot [ 1 ] = Rotate about the vector < 0.999 , - 0.007 , 0.046 > \\ by angle arc sin ( - 0.061 / cos ( - arc sin ( 0.046 ) ) \\ = - 0.0611 radians\end{matrix}$$

$$\begin{matrix}Rot [ 2 ] = Rotate about the vector < 0.007 , - 0.999 , 0 > \\ by angle - arc sin ( 0.046 ) \\ = - 0.046 radians\end{matrix}$$

$$\begin{matrix}Rot [ 3 ] = Rotate about the vector < 0 , 0 , 1 > \\ by angle - arc tan ( 0.999 / - 0.007 ) \\ = - 0.5778 radians\end{matrix}$$

$$\begin{matrix}Rot [ 4 ] = Rotate about the vector < 0 , 0 , 1 > \\ by angle arc tan ( - 0.756 / 0.642 ) \\ = - 0.8668 radians\end{matrix}$$

$$\begin{matrix}Rot [ 5 ] = Rotate about the vector < 0.654 , 0.756 , 0 > \\ by angle arc sin ( 0.128 ) \\ = 0.128 radians\end{matrix}$$

$$\begin{matrix}Rot [ 6 ] = Rotate about the vector < - 0.756 , 0.642 , 0.128 > \\ by angle - arc sin ( 0.033 / cos ( arc sin ( 0.128 ) ) ) \\ = - 0.0333 radians\end{matrix}$$

$$GO = Rot ( 0.999 , - 0.007 , 0.046 , - 0.061 ) \cdot Rot ( 0.007 , 0.999 , 0 , - 0.046 ) \cdot Rot ( 0 , 0 , 1 , - 1.5778 ) \cdot Rot ( 0 , 0 , 1 , - 0.8668 ) \cdot Rot ( 0.642 , 0.756 , 0 , 0.128 ) \cdot Rot ( - 0.756 , 0.642 , 0.128 , - 0.0333 )$$

$$GOR = LH \cdot GO \cdot LH$$

To find the local position 204, the initial local position vector, LPV 406 is set to the negative of the fiducial fixed position 404, assuming no initial model offset 405. LPV 406 is rotated by the right-handed transform, GOR, and added to the current fiducial position 304.

Fiducial Fixed Position =(−12.1, 1.3, 99.5) mm

Fiducial Position =(45.1, -15.2, 103.1) mm

LPV=—Fiducial Fixed Position

Local Position =Fiducial Position +LPV·GOR=(36.429, 5.867, 5.482) mm

The local axes, 201, 202, and 203 are computed by multiplying the corresponding global axes by GOR.

Local AxisX=Global AxisX·GOR=<−0.770, 0.632, 0.081>

Local AxisY=Global AxisY·GOR=<−0.638, −0.765, −0.095>

Local AxisZ=Global AxisZ·GOR=<0.002, −0.125, 0.992>

The catheter tip 905 is referenced back to the unrotated model by the matrix inverse of the transformation matrix, GOR, and it is seen to match the initial hand placement.

CatheterGlobal Axis=<0.593, −0.638, −0.490>

CatheterLocal Axis=CatheterGlobal Axis·GOR⁻¹=<−0.90, 0.156, -0.405 >

Given hand-accuracy, the Local Axis in the rotated patient matches the Global Axis in the unrotated patient: <−0.916, 0.090, −0.391 >

The vector from the local position origin 204 to the catheter's global position 904 is rotated back to global coordinates 100 for the catheter's local position 914 with respect to the un-rotated model.

$$Catheter global position = ( - 10.6 , 47 , - 4.3 ) mm$$

$$\begin{matrix}Catheter local position = ( Catheter global position - local position 204 ) \cdot \\ {GOR}^{- 1} \\ = ( 61.452 , - 0.533 , - 14.924 ) mm .\end{matrix}$$

Given hand-accuracy, the local position in the rotated patient matches the global position in the unrotated patient (61.1, -1.1, -14.6).

Many alterations and modifications may be made by those having ordinary skill in the art without departing from the spirit and scope of the invention. Therefore, it must be understood that the illustrated embodiments have been set forth only for the purposes of example and should not be taken as limiting the scope of the invention.

Therefore, it must be understood that the illustrated embodiment has been set forth only for the purposes of example and that it should not be taken as limiting the invention as defined by the following claims. For example, notwithstanding the fact that the elements of a claim are set forth below in a certain combination, it must be expressly understood that the invention includes other combinations of fewer, more or different elements, which are disclosed in above even when not initially claimed in such combinations. A teaching that two elements are combined in a claimed combination is further to be understood as also allowing for a claimed combination in which the two elements are not combined with each other, but may be used alone or combined in other combinations. The excision of any disclosed element of the invention is explicitly contemplated as within the scope of the invention.

The words used in this specification to describe the invention and its various embodiments are to be understood not only in the sense of their commonly defined meanings, but to include by special definition in this specification structure, material or acts beyond the scope of the commonly defined meanings. Thus if an element can be understood in the context of this specification as including more than one meaning, then its use in a claim must be understood as being generic to all possible meanings supported by the specification and by the word itself

The definitions of the words or elements of the following claims are, therefore, defined in this specification to include not only the combination of elements which are literally set forth, but all equivalent structure, material or acts for performing substantially the same function in substantially the same way to obtain substantially the same result. In this sense it is therefore contemplated that an equivalent substitution of two or more elements may be made for any one of the elements in the claims below or that a single element may be substituted for two or more elements in a claim. Although elements may be described above as acting in certain combinations and even initially claimed as such, it is to be expressly understood that one or more elements from a claimed combination can in some cases be excised from the combination and that the claimed combination may be directed to a subcombination or variation of a subcombination. Accordingly, the scope of the invention is limited only by the claims and equivalents thereto.

What is claimed is:

1. A method for determining the position and orientation of an invasive medical device having a magnetic tip with a defined magnetic axis in a patient comprising:
obtaining sensor measured data from a plurality of magnetic field sensors, said magnetic field sensors configured to sense a magnetic field produced by said tip;
computing first sensor range estimates using, at least in part said sensor measured data, and storing said first sensor range estimate as previous sensor range estimates;
computing a first position estimate of the tip using, at least in part said first sensor range estimates, and saving said first position estimate as a previous position estimate;
computing a first orientation estimate a magnetic axis of the tip using, at least in part said first sensor range estimates, and saving said first orientation estimate as a previous orientation estimate;
computing revised sensor range estimates using, at least in part, said previous orientation estimate, and storing said revised sensor range estimate as said previous sensor range estimates;
computing a revised position estimate of the tip using, at least in part, said previous sensor range estimates, and saving said revised position estimate as said previous position estimate;
computing a revised orientation estimate of a magnetic axis of the tip using, at least in part, said revised sensor range estimates, and saving said revised orientation estimate as said previous orientation estimate; and
repeating the process of computing revised sensor range estimates, computing a revised position estimate, and computing a revised orientation estimate until a desired accuracy is obtained for said revised position estimate and said revised orientation estimate.

2. The method of claim 1, where computing the revised orientation of the magnetic axis of the tip comprises:
computing a first sensor-to-tip vector using the previous position estimate and a sensor position of a first sensor;
obtaining a first magnetic field vector measured by said first sensor;
computing a vector describing a first magnetic plane, at least in part by, using a cross product of the first sensor-to-tip vector with the first magnetic field vector;
computing a second sensor-to-tip vector using the previous position estimate and a sensor position of a second sensor;
obtaining second magnetic field vector measured by said second sensor;
computing a vector describing a second magnetic plane, at least in part, by using a cross product of the second sensor-to-tip vector with the second magnetic field vector; and
computing said revised orientation, at least in part by, computing an angular orientation of an intersection of the first magnetic plane and the second magnetic plane.

3. The method of claim 2, further comprising computing an average obtained by first computing said revised orientation when said first sensor and said second sensor comprise a first pair of sensors chosen from a plurality of sensors and then computing said revised orientation when said first sensor and said second sensor comprise a second pair of sensors chosen from said plurality of sensors.

4. The method of claim 3, wherein said first sensor and said second sensor comprise spatially-adjacent sensors in said plurality of sensors.

5. The method of claim 1, wherein said first sensor range estimates are computed using a spherical magnetic field pattern for said tip.

6. The method of claim 1, wherein said first sensor range estimates are computed using an non-spherical magnetic field pattern for said tip.

7. The method of claim 1, wherein said first sensor range estimates are computed using a magnetic field pattern for said tip based on measured field patterns of said tip.

8. The method of claim 1, wherein said first range estimates are computed, at least in part by, measuring a sensor angle for each sensor.

9. A method for correlating a fiducial alignment with a medical device position detection system and model imaging system comprising:
establishing a global coordinate system, GX, GY, and GZ, relative to a magnetic coil array;
establishing a local coordinate system, LX, LY, and LZ, relative to a patient;
establishing a current fiducial coordinate system, FX, FY, and FZ, relative a movable and orientable fiducial sensor, the current fiducial coordinate system being referenced to the global coordinate system;
determining position offset vector, MO to align the global and local coordinate systems;
establishing a fixed fiducial coordinate system, FFX, FFY, and FFZ, related to the aligned global coordinate and local coordinate systems by a local position vector, LPV;
rotating the fixed fiducial coordinate system to the current fiducial coordinate system using a global orientation matrix GO; and
translating virtual models from the global coordinate system to the local coordinate system using a global positioning matrix, GP; and
rotating virtual models from the global coordinate system to the local coordinate system using a global orientation matrix, GO.

10. The method of claim 9, comprising translating local catheter position and orientation in a rotated patient to global catheter position and orientation in an unrotated patient.

11. A method for guiding, steering, and advancing an invasive medical device having a magnetic tip in a patient in which cardiac and pulmonary movement is occurring comprising:
adjusting a physical position of a virtual tip of the medical device;
encoding a change in a virtual tip position;
measuring an actual tip position of the medical device with a detector system;
communicating the virtual tip and actual tip position to a control system;
generating servo-system commands in the control system to move the magnetic tip from the actual tip position toward the encoded changed virtual tip position;
communicating the servo-system commands to a servo system control apparatus;
adjusting a force applied to the magnetic tip by one or more electromagnet clusters in response to the servo-system commands;
sensing a new actual position of the magnetic tip by the detector system;
sensing a position of a plurality of fiducial markers;
constructing an image of the new actual position of the magnetic tip relative to the position of the fiducial markers;
synchronizing and superimposing of an image of the magnetic tip produced by another imaging modality to the constructed image;
providing feedback to the servo system control apparatus; and
updating the image of the actual magnetic tip position in relation to the patient's internal body structures.

12. An apparatus for controlling the movement of a magnetic surgical tool inside a body of a patient, comprising:
a magnetic field source configured to produce a magnetic field to produce movement and orientation of a magnetic surgical tool;
a plurality of magnetic field sensors positioned to sense a magnetic field produced by said magnetic surgical tool:
a system controller for controlling said magnetic field source, said system controller configured to obtain sensor data from said plurality of magnetic field sensors and compute a position and orientation of said magnetic surgical tool at least in part by using said sensor data to compute estimates of said position and orientation, said system controller further configured to iteratively refine said estimates by using previously computed estimates of said position and orientation, said system controller configured to iterate calculation said estimates until a desired accuracy is achieved.

13. The apparatus of claim 12, wherein said controller uses said estimates of position and orientation to control said magnetic field source to provide closed-loop control of said magnetic surgical tool.

14. The apparatus of claim 12, further comprising one or more fiducial sensors to sense a position of a fiducial marker, said system controller configured to transform said estimates of position and orientation into transformed position and orientation using at least in part data from said one or more fiducial sensors.

15. The apparatus of claim 12, wherein said servo system comprises a correction factor that compensates for a dynamic position of an organ, thereby offsetting a response of said magnetic surgical tool to said magnetic field such that said magnetic surgical tool moves in substantial unison with said organ.

16. The apparatus of claim 12, wherein said correction factor is generated from an auxiliary device that provides correction data concerning said dynamic position of said organ, and wherein when said correction data are combined with measurement data derived from said sensory apparatus to offset a response of said servo system so that said magnetic surgical tool moves substantially in unison with said organ.

17. The apparatus of claim 16, wherein said auxiliary device comprises at least one of an X-ray device, an ultrasound device, and a radar device.

18. The apparatus of claim 12, wherein data from a haptic joystick is provided to said system controller.

19. The apparatus of claim 12, wherein said magnetic field sensors comprises Hall effect sensor.

20. The apparatus of claim 12, wherein said system controller controls an amplitude and phase of current through a plurality of electromagnet coils in said magnetic field source.

21. The apparatus of claim 12, wherein a haptic joystick provides tactile feedback to an operator.

22. The apparatus of claim 12, wherein a haptic joystick provides tactile feedback to an operator according to a position error between an actual position of said magnetic surgical tool and a desired position of said magnetic surgical tool.

23. The apparatus of claim 12, wherein said system controller causes said magnetic surgical tool to follow movements of said haptic joystick.

24. The apparatus of claim 12, wherein a correction input is generated by an auxiliary device that provides correction data concerning a dynamic position of an organ, and wherein said correction data are combined with measurement data to offset a response of said control system so that said magnetic surgical tool moves substantially in unison with said organ.

25. The apparatus of claim 24, wherein said auxiliary device comprises at least one of an X-ray device, an ultrasound device, and a radar device.
